(19) United States
(12) Patent Application Publication
(10) Pub. No.: US 2018/0369264 A1
(43) Pub. Date: Dec. 27, 2018

(54) COMBINATION OF A CARDIAC STEROID AND AN AKT INHIBITOR FOR THE TREATMENT OF CARDIOVASCULAR DISEASES AND DISORDERS

(71) Applicant: YISSUM RESEARCH DEVELOPMENT COMPANY OF THE HEBREW UNIVERSITY OF JERUSALEM LTD., Jerusalem (IL)

(72) Inventors: David LICHTSTEIN, Shoeva (IL); Nahum BUZAGLO, Tel-Aviv (IL)

(21) Appl. No.: 16/063,611

(22) PCT Filed: Dec. 20, 2016
(86) PCT No.: PCT/IL2016/051360

§ 371 Date: Jun. 18, 2018

Related U.S. Application Data
(60) Provisional application No. 62/387,209, filed on Dec. 24, 2015.

(51) Int. Cl. A61K 31/7048; A61K 31/585; A61P 9/10

(52) U.S. Cl. CPC A61K 31/7048; A61K 31/585; A61K 31/675; A61K 2300/00; A61P 9/10

(57) ABSTRACT

The present invention relates to pharmaceutical combinations for the treatment of cardiovascular diseases and disorders. More particularly, the invention relates to pharmaceutical combinations comprising a cardiac steroids CS and at least one PI3K/Akt/m TOR inhibitor. The compositions of the invention may particularly be used for reducing the CS dose administered to a subject suffering from a cardiovascular disease or disorders, thereby reducing the side effects associated with CS therapy. The invention further provides methods of treatment of such diseases and disorders using the pharmaceutical combinations.

### FIELD OF THE INVENTION

The present invention relates to pharmaceutical combinations for the treatment of cardiovascular diseases or disorders. More particularly, the invention relates to pharmaceutical combinations comprising a cardiac steroid (CS) and at least one PI3K/Akt/mTOR inhibitor.

### BACKGROUND OF THE INVENTION

Cardiovascular disease represents the leading cause of morbidity and death in developed countries. Coronary heart disease (CHD), which is the single largest cause of cardiovascular disease, is the narrowing of arteries over time caused by atherosclerotic plaques or the acute occlusion of the coronary artery by thrombosis, both of which lead to possible myocardial infarction (MI) and the eventual development of heart failure.

The treatment of heart failure most frequently requires a combination of medications. The drugs in use include angiotensin-converting enzyme (ACE) inhibitors (enalapril (Vasotec), lisinopril (Zestril) and captopril (Capoten)); angiotensin II receptor blockers (losartan (Cozaar) and valsartan (Diovan)); Beta blockers (carvedilol (Coreg), metoprolol (Lopressor) and bisoprolol (Zebeta)); Diuretics (furosemide (Lasix)); Aldosterone antagonists (spironolactone and eplerenone) and Cardiac steroids (Digoxin (Lanoxin)). Among all available drugs, digoxin is the only one that directly increases the force of heart muscle contractions.

Cardiac steroids (CS), containing cardenolides and bufadienolides, such as digoxin, ouabain and bufalin, are extracted from various plants and toad skin. The CSs are used to increase the force of contraction of heart muscle and regulate its rhythm in heart failure and arrythmogenic patients, respectively. Nevertheless, the therapeutic window for CS is extremely small. Whereas plasma concentration of about 1 nM digoxin is considered beneficial, significant signs of toxicity are observed already at 3 nM. The advantage of using CS in a clinical setting is still debatable. A comprehensive study testing the beneficial effects of digoxin (Digitalis Investigation Group, DIG study, https://clinicaltrials.gov/ct2/show/NCT00000476) showed that digoxin did not reduce overall mortality, but rather the rate of hospitalization, both overall and for worsening heart failure. Recent studies, however, have shown that heart failure in patients treated with digoxin was associated with lower all-cause mortality and hospitalization than in patients in the placebo group, advocating the use of this drug, despite its small therapeutic index.

The regularly used CS in the clinic is digoxin. The drug causes numerous side effects the most frequent are dizziness, fainting, changes in heart beat rate and arrhythmias. Less frequent side effects include blood in the urine or stools, severe stomach pain and neurological symptoms such as anxiety, confusion and depression. These side effects impede the use of CS and points to the importance of increasing the therapeutic window of these drugs. Despite the above, total sale of Digoxin is $40,000,000 annually and 8 companies (Novartis; Glaxo; Mano Pharmaceuticals; Cadila Pharmaceuticals; Zydus Gadila; Samarth Pharma; Sanofi Synthelabo) manufacture generic Digoxin.

The only established receptor for CS is the ubiquitous, plasma membrane, sodium-potassium-dependent adenosine triphosphatase (Na⁺, K⁺-ATPase). This transporter plays a crucial role in maintaining the Na⁺ and K⁺ gradients across the plasma membrane. The binding of CS to a specific site located in the extracellular loop of the alpha subunit of Na⁺, K⁺-ATPase causes the inhibition of ATP hydrolysis and ion transport by the pump, reducing Na⁺ and K⁺ gradients across the plasma membrane and, as a result, affecting numerous cell functions. These effects of CS on ionic gradients are the common explanation for the mechanism underlying the CS-induced increase in the force of contraction of heart muscle.

Studies in the past decade have demonstrated that in addition to pumping ions, the Na⁺, K⁺-ATPase is engaged in the assembly of multiple protein complexes into functional micro-domains that transmit signals into the cell. The interaction of CS, at nM or sub nM concentrations, with Na⁺, K⁺-ATPase activates signal transduction cascades of the Src tyrosine kinase/MAP-kinase and PI3K1A/PDK/Akt pathways in different cell types, including cardiomyocytes, smooth muscle, neuronal and epithelial cells. This CS-induced signal transduction activation was shown to be involved in several physiological processes, including the regulation of gene expression, cell viability, differentiation and smooth muscle contraction.

Akt, also designated Protein kinase B (PKB), is a cytosolic serine/threonine kinase that promotes cell survival by inactivation of targets of the apoptotic pathways, and is implicated in the execution of many other cellular processes including: cell proliferation, angiogenesis, glucose metabolism, protein translation, and gene transcription, all are mediated by extracellular and intracellular signals.

In many cancers Akt is overexpressed and has central role in cancer progression and cancer cell survival. These observations made AKT an attractive target for cancer therapy. The MK-2206 is a potent allosteric inhibitor of AKT with anti-proliferative activity alone and in combination with other agents in human cancer cell lines, including breast, ovarian, lung, and prostate cancer. Currently, 209 clinical studies are being conducted (Phase I, II and III) to test the beneficial effect of AKT inhibitors in cancer treatment. These are being conducted by the major pharmaceutical companies including Merck, Pfizer, GlaxoSmithKline, Abbott Laboratories, Novartis and more.

It is therefore an object of the invention to provide a combination treatment comprising a cardiac steroid (CS) and at least one inhibitor of the PI3K/Akt/mTOR signaling pathway.

Another object of the invention is to provide the use of these combinations for the treatment of cardiovascular disorders.

It is still a further object of the invention to provide a pharmaceutical combination for use in reducing side effects associated with CS therapy.

These and other objects of the invention will become apparent as the description proceeds.

### SUMMARY OF THE INVENTION

In one aspect, the present invention provides a pharmaceutical combination comprising a cardiac steroid (CS) and at least one PI3K/Akt/mTOR inhibitor.

In one embodiment, the CS is selected from:
3-(alpha-L-Rhamnopyranosyloxy)-1beta,5beta,11alpha,14,19-pentahydroxy-5beta-card-20(22)-enolide (ouabain);
4-[(3 S,5R,8R,9S,10S,12R,13 S, 14S)-3-[(2S,4S,5R,6R)-5-[(2S,4S,5R,6R)-5-[(2S,4S,5R,6R)-4,5-dihydroxy-6-methyl-oxan-2-yl]oxy-4-hydroxy-6-methyl-oxan-2-yl]oxy-4-hydroxy-6-methyl-oxan-2-yl]oxy-12,14-dihydroxy-10,13-dimethyl-1,2,3,4,5,6,7,8,9,11,12,15,16,17-tetradecahydrocyclopenta[a]phenanthren-17-yl]-5H-furan-2-one (digoxin);
5-[(3 S,5R,8R,9S,10S,13R,14S,17R)-3,14-dihydroxy-10,13-dimethyl-1,2,3,4,5,6,7,8,9,11,12,15,16,17-tetradecahydrocyclopenta[a]phenanthren-17-yl]pyran-2-one (bufalin);
(3β,5β)-3-{[3-O-Acetyl-2,6-dideoxy-β-D-ribo-hexopyranosyl-(1->4)-2,6-dideoxy-β-D-ribo-hexopyranosyl-(1->4)-2,6-dideoxy-β-D-ribo-hexopyranosyl]oxy}-14-hydroxycard-20(22)-enolide (acetyldigitoxin);
(3β,5β,12β)-3-{[3-O-Acetyl-2,6-dideoxy-β-D-ribo-hexopyranosyl-(1->4)-2,6-dideoxy-β-D-ribo-hexopyranosyl-(1->4)-2,6-dideoxy-β-D-ribo-hexopyranosyl]oxy}-12,14-dihydroxycard-20(22)-enolide (acetyldigoxin);
5,14-dihydroxy-3-(5-hydroxy-4-methoxy-6-methyloxan-2-yl)oxy-13-methyl-17-(5-oxo-2H-furan-3-yl)-2,3,4,6,7,8,9,11,12,15,16,17-dodecahydro-1H-cyclopenta[a]phenanthrene-10-carbaldehyde (cymarin);
(3β,5β)-3-{[2,6-Dideoxy-β-D-ribo-hexopyranosyl-(1->4)-2,6-dideoxy-β-D-ribo-hexopyranosyl-(1->4)-2,6-dideoxy-β-D-ribo-hexopyranosyl]oxy}-14-hydroxycard-20(22)-enolide (digitoxin);
(3β,5β)-3,14-Dihydroxycard-20(22)-enolide (digitoxigenin);
(3β,5β,12β)-3,12,14-Trihydroxycard-20(22)-enolide (digoxigenin);
(3β,5β,12β)-3-{[2,6-Dideoxy-4-O-methyl-β-D-ribo-hexopyranosyl-(1->4)-2,6-dideoxy-β-D-ribo-hexopyranosyl-(1->4)-2,6-dideoxy-β-D-ribo-hexopyranosyl]oxy}-12,14-dihydroxycard-20(22)-enolide (metildigoxin);
(3 S,5 S,8R,9S,10 S,13R,14S,17R)-5,14-Dihydroxy-13-methyl-17-(5-oxo-2,5-dihydro-3-furanyl)-3-{[(2R,3R,4R,5R,6R)-4,5,6-trihydroxy-3-{[(2S,3R,4S,5 S,6R)-3,4,5-trihydroxy-6-(hydroxymethyl)tetrahydro-2H-pyran-2-yl]oxy}tetrahydro-2H-pyran-2-yl]oxy}hexadecahydro-1 OH-cyclopenta[a]phenanthrene-10-carbaldehyde (neoconvalloside);
(3β,5β)-3-{[2,6-Dideoxy-4-O-(β-D-glucopyranosyl)-3-O-methyl-β-D-ribo-hexopyranosyl]oxy}-5,14-dihydroxy-19-oxocard-20(22)-enolide (k-strophanthin);
(3β,5β)-3,5,14-Trihydroxy-19-oxocard-20(22)-enolide (k-strophanthidin);
5-[(5R,8R,9S,10S,13S,14S,17S)-10,13-dimethyl-2,3,4,5,6,7,8,9,11,12,14,15,16,17-tetradecahydro-1H-cyclopenta[a]phenanthren-17-yl]pyran-2-one (bufadienolide);
5-[(3 S,8R,9S,1 OR, 13R, 14S,17R)-14-Hydroxy-10,13-dimethyl-3-((2R,3R,4R,5R,6R)-3,4,5-trihydroxy-6-methyltetrahydro-2H-pyran-2-yloxy)-2,3,6,7,8,9,10,11,12,13,14,15,16,17-tetradecahydro-1H-cyclopenta[a]phenanthren-17-yl]-2H-pyran-2-one (proscillaridin);
(1β,3β,5β,11α)-1,3,5-(ethylidynetris(oxy)-11,14-dihydroxy-12,19-dioxobufa-20,22-dienolide (daigremontianin); and
(3β,5β,15β)-3,5-Dihydroxy-14,15-epoxybufa-20,22-dienolide (marinobufagenin).

In another embodiment, the PI3K/Akt/mTOR inhibitor in the present invention is an Akt inhibitor, which is selected from:
8-[4-(1-Aminocyclobutyl)phenyl]-9-phenyl[1,2,4]triazolo[3,4-f][1,6]naphthyridin-3(2H)-one dihydrochloride (MK-2206 2HCl);
1,1-dimethyl-4 [(octadecyloxy)hydroxyphosphinyl]oxy]-piperidinium inner salt, KRX-0401 (perifosine);
4-[2-(4-amino-1,2,5-oxadiazol-3-yl)-1-ethyl-7-[[(3 S)-piperidin-3-yl]methoxy]imidazo[4,5-c]pyridin-4-yl]-2-methylbut-3-yn-2-ol (GSK690693);
(2S)-2-(4-Chlorophenyl)-1-{4-[(5R,7R)-7-hydroxy-5-methyl-6,7-dihydro-5H-cyclopenta[d]pyrimidin-4-yl]-1-piperazinyl}-3-(isopropylamino)-1-propanone (GDC-0068, ipatasertib);
4-amino-N-[(1 S)-1-(4-chlorophenyl)-3-hydroxypropyl]-1-(7H-pyrrolo[2,3-d]pyrimidin-4-yl)piperidine-4-carboxamide (AZD5363);
2-amino-8-[4-(2-hydroxyethoxy)cyclohexyl]-6-(6-methoxypyridin-3-yl)-4-methylpyrido[2,3-d]pyrimidin-7-one (PF-04691502);
4-(4-chlorophenyl)-4-[4-(1H-pyrazol-4-yl)phenyl]piperidine (AT7867);
5-Methyl-1-(β-D-ribofuranosyl)-1,5-dihydro-1,4,5,6,8-pentaazaacenaphthylen-3-amine (Triciribine);
4-(4-Chlorobenzyl)-1-(7H-pyrrolo[2,3-d]pyrimidin-4-yl)-4-piperidinamine (CCT128930);
(2S)-1-[5-(3-methyl-2H-indazol-5-yl)pyridin-3-yl]oxy-3-phenylpropan-2-amine (A-674563);
4-dodecyl-N-(1,3,4-thiadiazol-2-yl)benzenesulfonamide (PHT-427);
3-[1-[[4-(7-phenyl-3H-imidazo[4,5-g]quinoxalin-6-yl)phenyl]methyl]piperidin-4-yl]-1H-benzimidazol-2-one (Akti-1/2);
N-[(2S)-1-amino-3-(3-fluorophenyl)propan-2-yl]-5-chloro-4-(4-chloro-2-methylpyrazol-3-yl)thiophene-2-carboxamide (GSK2110183, afuresertib);
(1 S)-2-amino-1-(4-chlorophenyl)-1-[4-(1H-pyrazol-4-yl)phenyl]ethanol);
Miltefosine (hexadecyl 2-(trimethylazaniumyl)ethyl phosphate (AT13148);
2-(4-hydroxy-3-prop-2-enylphenyl)-4-prop-2-enylphenol (Honokiol);
2,6,7,8,9,10-hexahydro-10-[(2-methylphenyl)methyl]-7-(phenylmethyl)-imidazo[1,2-a]pyrido[4,3-d]pyrimidin-5(3H)-one (TIC10 Analogue);
2,4,6,7,8,9-hexahydro-4-[(2-methylphenyl)methyl]-7-(phenylmethyl)-imidazo[1,2-a]pyrido[3,4-e]pyrimidin-5(1H)-one (TIC10); and
ethyl-3-aminobenzoate methanesulfonate salt (MS-222).

In one specific embodiment, the pharmaceutical combination of the invention further comprises at least one additional therapeutic agent.

In another aspect, the present invention provides a pharmaceutical combination comprising a CS and at least one PI3K/Akt/mTOR inhibitor for use in treating a cardiovascular disease or disorder.

In a further aspect, the present invention provides a pharmaceutical combination comprising a CS and at least one PI3K/Akt/mTOR inhibitor for simultaneous, sequential or separate use in treating a cardiovascular disease or disorder.

In a further aspect, the present invention provides the use of a CS and at least one PI3K/Akt/mTOR inhibitor for the manufacture of a medicament for the treatment of a cardiovascular disease or disorder.

In a further aspect, the present invention provides a PI3K/Akt/mTOR inhibitor for use in potentiating the activity of a cardiac steroid.

In a still further aspect, the present invention provides a method of treating a cardiovascular disease or disorder by administering a combination of a CS and at least one PI3K/Akt/mTOR inhibitor to a subject in need thereof.

In another aspect, the present invention provides a method for improving efficacy of the treatment of a cardiovascular disease or disorder with at least one PI3K/Akt/mTOR inhibitor comprising administering a combination comprising a CS and at least one PI3K/Akt/mTOR inhibitor to a subject in need thereof.

In a still further aspect, the present invention provides a kit comprising: (a) a first container with a CS; (b) a second container with PI3K/Akt/mTOR inhibitor; optionally (c) a third container with a third pharmaceutical formulation; and optionally (d) label or package insert with instructions for treating a cardiovascular disease or disorder.

In one specific embodiment, the CS and the PI3K/Akt/mTOR inhibitor of the kit are provided as different dosage forms, each one in a suitable carrier.

### BRIEF DESCRIPTION OF THE DRAWINGS

FIGS. 1A-1I show in-vivo heart contractility measurements and their validation in zebrafish larvae.

FIG. 1A is a schematic of zebrafish larva at 72 hours post fertilization (hpf). The single atrium and ventricle that lie anteriorly on the ventral surface of the fish is marked by an arrow.

FIG. 1B shows the endomyocardial border at the end of systole, at high magnification, to define the ventricular area.

FIG. 1C shows the endomyocardial border at the end of diastole, at high magnification, to define the ventricular area.

FIG. 1D shows midventricular short axis (SA) and long axis (LA) at the end of systole.

FIG. 1E shows midventricular short axis (SA) and long axis (LA) at the end of diastole.

FIG. 1F shows the Fractional Area Change (FAC) following treatment of 1 and 10 μM carbachol (Carb) or adrenaline (Adr) for 90 minutes. *Significantly different from control, P<0.05.

FIG. 1G shows the Ejection Fraction (EF) following treatment of 1 and 10 μM carbachol or adrenaline for 90 minutes. *Significantly different from control, P<0.05.

FIG. 1H shows the heart rate (HR) following treatment of 1 and 10 μM carbachol or adrenaline for 90 minutes. *Significantly different from control, P<0.05.

FIG. 1I shows the calculated Cardiac Output (CO) following treatment of 1 and 10 M carbachol or adrenaline for 90 minutes. *Significantly different from control, P<0.05.

FIGS. 2A-2D show the effect of ouabain on zebrafish heart contractility.

FIG. 2A shows the FAC following treatment of 0.025-0.4 nM ouabain, for 90 minutes. *Significantly higher than the control, P<0.05.

FIG. 2B shows the EF following treatment of 0.025-0.4 nM ouabain, for 90 minutes. *Significantly higher than the control, P<0.05.

FIG. 2C shows the HR following treatment of 0.025-0.4 nM ouabain, for 90 minutes. *Significantly higher than the control, P<0.05.

FIG. 2D shows the CO following treatment of 0.025-0.4 nM ouabain, for 90 minutes. *Significantly higher than the control, P<0.05.

FIGS. 3A-3D show the effect of digoxin on zebrafish heart contractility.

FIG. 3A shows the FAC following treatment of 0.1-1000 nM digoxin, for 90 minutes. *Significantly higher than the control, P<0.05.

FIG. 3B shows the EF following treatment of 0.1-1000 nM digoxin, for 90 minutes. *Significantly higher than the control, P<0.05.

FIG. 3C shows the HR following treatment of 0.1-1000 nM digoxin, for 90 minutes. *Significantly lower than the control, P<0.05.

FIG. 3D shows the CO following treatment of 0.1-1000 nM digoxin, for 90 minutes. *Significantly higher than the control, P<0.05.

FIGS. 4A-4D show the effect of bufalin on zebrafish heart contractility.

FIG. 4A shows the FAC following treatment of 0.01-10 nM bufalin, for 90 minutes. *Significantly higher than the control, P<0.05.

FIG. 4B shows the EF following treatment of 0.01-10 nM bufalin, for 90 minutes. *Significantly higher than the control, P<0.05.

FIG. 4C shows the HR following treatment of 0.01-10 nM bufalin, for 90 minutes. *Significantly lower than the control, P<0.05.

FIG. 4D shows the CO following treatment of 0.01-10 nM bufalin, for 90 minutes. *Significantly higher than the control, P<0.05.

FIGS. 5A-5D show the effect of CS on zebrafish Accordion mutant (acc) heart contractility.

FIG. 5A shows the FAC following treatment of 1 nM ouabain, digoxin, or bufalin, for 90 minutes.

FIG. 5B shows the EF following treatment of 1 nM ouabain, digoxin, or bufalin, for 90 minutes.

FIG. 5C shows the HR following treatment of 1 nM ouabain, digoxin, or bufalin, for 90 minutes.

FIG. 5D shows the CO following treatment of 1 nM ouabain, digoxin, or bufalin, for 90 minutes.

Abbreviations: C (control); O (ouabain); D (digoxin); B (bufalin).

FIGS. 6A-6D show the effect of ouabain on zebrafish acc mutant heart contractility.

FIG. 6A shows the FAC following treatment of 0.01-10 nM ouabain, for 90 minutes.

FIG. 6B shows the EF following treatment of 0.01-10 nM ouabain, for 90 minutes.

FIG. 6C shows the HR following treatment of 0.01-10 nM ouabain, for 90 minutes.

FIG. 6D shows the CO following treatment of 0.01-10 nM ouabain, for 90 minutes.

FIGS. 7A-7D show the effect of CS on extracellular signal-regulated kinases (ERK) and Akt phosphorylation in adult zebrafish heart.

FIG. 7A shows the levels of phosphorylated Akt (pAkt) and total Akt (tAkt) following treatment of 1 μM ouabain, digoxin, or bufalin, for 5 minutes.

FIG. 7B shows Akt phosphorylation state following treatment of 1 μM ouabain, digoxin, or bufalin, for 5 minutes. *Significantly higher than the control, P<0.05.

FIG. 7C shows the levels of phosphorylated ERK (pERK) and total ERK (tERK) following treatment of 1 μM ouabain, digoxin, or bufalin, for 5 minutes.

FIG. 7D shows ERK phosphorylation state following treatment of 1 μM ouabain, digoxin, or bufalin, for 5 minutes. *Significantly higher than the control, P<0.05.

Abbreviations: C (control); O (ouabain); D (digoxin); B (bufalin).

FIGS. 8A-8D show the effect of CS on ERK and Akt phosphorylation in adult zebrafish acc mutant heart.

FIG. 8A shows the levels of phosphorylated Akt (pAkt) and total Akt (tAkt) following treatment of 1 μM ouabain, digoxin, or bufalin, for 5 minutes.

FIG. 8B shows Akt phosphorylation state following treatment of 1 μM ouabain, digoxin, or bufalin, for 5 minutes. *Significantly higher than the control, P<0.05.

FIG. 8C shows the levels of phosphorylated ERK (pERK) and total ERK (tERK) following treatment of 1 μM ouabain, digoxin, or bufalin, for 5 minutes.

FIG. 8D shows ERK phosphorylation state following treatment of 1 μM ouabain, digoxin, or bufalin, for 5 minutes. *Significantly higher than the control, P<0.05.

Abbreviations: C (control); O (ouabain); D (digoxin); B (bufalin).

FIGS. 9A-9D show the effect of the ERK inhibitor U0126 (1,4-diamino-2,3-dicyano-1,4-bis[2-aminophenylthio]butadiene) and Src tyrosine kinase (Src) inhibitor (pp2) on CS-induced increase in heart contractility in zebrafish larvae.

FIG. 9A shows the FAC following treatment of 1 μM U0126 for 30 minutes, and 1 nM oubain, digoxin, or bufalin for additional 90 minutes. *Significantly higher than the control, P<0.05.

FIG. 9B shows the EF following treatment of 1 μM U0126 for 30 minutes, and 1 nM oubain, digoxin, or bufalin for additional 90 minutes. *Significantly higher than the control, P<0.05.

FIG. 9C shows the FAC following treatment of 50 nM pp2 for 30 minutes, and 1 nM oubain, digoxin, or bufalin for additional 90 minutes. *Significantly higher than the control, P<0.05.

FIG. 9D shows the EF following treatment of 50 nM pp2 for 30 minutes, and 1 nM oubain, digoxin, or bufalin for additional 90 minutes. *Significantly higher than the control, P<0.05.

Abbreviations: C (control); O (ouabain); D (digoxin); B (bufalin).

FIGS. 10A-10D show the effect of Akt inhibitor (MK-2206) on CS-induced increase in heart contractility in wild-type (wt) and in acc mutant zebrafish larvae.

FIG. 10A shows the FAC of acc mutants following treatment of 10 nM MK-2206 for 30 minutes, and 1 nM oubain, digoxin, or bufalin for additional 90 minutes. *Significantly higher than the control, P<0.05.

FIG. 10B shows the EF of acc mutants following treatment of 10 nM MK-2206 for 30 minutes, and 1 nM oubain, digoxin, or bufalin for additional 90 minutes. *Significantly higher than the control, P<0.05.

FIG. 10C shows the FAC of wt larvae following treatment of 10 nM MK-2206 for 30 minutes, and 1 nM oubain, digoxin, or bufalin for additional 90 minutes. *Significantly higher than the control, P<0.05; ^{#}Significantly higher than the 1 nM CS alone, P<0.05.

FIG. 10D shows the EF of wt larvae following treatment of 10 nM MK-2206 for 30 minutes, and 1 nM oubain, digoxin, or bufalin for additional 90 minutes. *Significantly higher than the control, P<0.05; ^{#}Significantly higher than the 1 nM CS alone, P<0.05.

Abbreviations: C (control); O (ouabain); D (digoxin); B (bufalin); MK (MK-2206).

FIGS. 11A-11F show the effect of the ERK inhibitor, PD98059 (2′-amino-3′-methoxyflavone) and Akt inhibitor (MK-2206) on CS-induced increase in primary adult zebrafish cardiomyocyte contractility.

FIG. 11A is a representative twitch of % shortening of cells stimulated at 0.5 Hz following exposure to 0.1 nM ouabain with or without 10 nM PD98059 for 20 minutes.

FIG. 11B shows quantification of the data presented in FIG. 11A as % of control. *Significantly higher than control, P<0.05.

FIG. 11C is a representative twitch of % shortening of cells stimulated at 0.5 Hz following exposure to 0.1 nM ouabain, with or without 1 nM MK-2206, for 20 minutes.

FIG. 11D shows quantification of the data presented in FIG. 11B as % of control. *Significantly higher than control, P<0.05; ^{#}Significantly higher than 0.1 nM ouabain alone, P<0.05.

FIG. 11E is a representative twitch of % shortening of acc mutant-derived cells stimulated at 0.5 Hz following exposure to 0.1 nM ouabain with or without 1 nM MK-2206 for 20 minutes.

FIG. 11F shows quantification of the data presented in FIG. 11E as % of control. *Significantly higher than control, P<0.05 Abbreviations: C (control); PD (PD98059); O (ouabain); MK (MK-2206).

FIGS. 12A-12D show the effect of ouabain in the presence or absence of Akt inhibitor (MK-2206) on Ca²⁺ transients in zebrafish isolated adult heart.

FIG. 12A is a representative Ca²⁺ transients in wt zebrafish in control and following 200 μM ouabain administration.

FIG. 12B is a representative of Ca²⁺ transients in acc mutant zebrafish in control and following 200 μM ouabain administration.

FIG. 12C is a representative of Ca²⁺ transients in wt zebrafish following 200 μM ouabain administration in the presence of 1 nM MK-2206.

FIG. 12D is a representative of Ca²⁺ transients in acc mutant zebrafish following 200 μM ouabain administration in the presence of 1 nM MK-2206.

Abbreviations: C (control); O (ouabain); MK (MK-2206).

FIGS. 13A-13F show the effects of ouabain, Akt inhibitor (MK-2206) and their combination on heart contractility in LAD-ligated rats.

FIG. 13A shows the difference between SF prior and post LAD-ligation (ASF), following i.p. injection of saline (0.5 ml/kg/day, control), ouabain (0.8-8 mg/kg/day) at 1, 3, 6, and 10 days post LAD-ligation. ^{#}Significantly lower than baseline, P<0.05; *Significantly higher than 24 hours post LAD-ligation, P<0.05.

FIG. 13B shows ASF following i.p. injection of saline (0.5 ml/kg/day, control), ouabain (0.8 mg/kg/day), MK-2206 (12 mg/kg/day) or combination of the two drugs at 1, 3, 6, and 10 days post LAD-ligation. ^{#}Significantly lower than baseline, P<0.05; *Significantly higher than 24h post LAD-ligation, P<0.05; ^{&}Significantly higher than the level in control at the same day, P<0.05.

FIG. 13C is a representative staining for fibrosis (by Masson's Trichrome) and collagen (by Aniline Blue) of hearts treated with saline (0.5 ml/kg/day, control), ouabain (0.8 mg/kg/day), MK-2206 (12 mg/kg/day) or combination of the two drugs at 10 days post LAD-ligation.

FIG. 13D shows the quantification of C, presented as the ratio between the scar area and the total heart area. *Significantly lower than the value in control, P<0.05.

FIG. 13E shows the HR following treatment of ouabain (0.8-8 mg/kg/day) at 1, 3, 6, and 10 days post LAD-ligation.

FIG. 13F shows the HR following treatment of ouabain (0.8 mg/kg/day), MK-2206 (12 mg/kg/day) or combination of the two drugs at 1, 3, 6, and 10 days post LAD-ligation.

Abbreviations: T (time post LAD); C (control); O (ouabain); MK (MK-2206); MIX (ouabain+MK-2206).

### DETAILED DESCRIPTION OF THE INVENTION

The present invention relates to a pharmaceutical combination comprising a cardiac steroid (CS) and at least one inhibitor of the PI3K/Akt/mTOR signaling pathway.

The term “combination” as used herein refers to either a fixed combination in one dosage unit form, or a number of therapeutic agents (also designated herein as “active ingredients”) for the combined administration where the agents may be administered independently at the same time or separately within time intervals, especially where these time intervals allow the therapeutic agents of the combination to show a synergistic effect.

The term “pharmaceutical combination” as used herein refers to a product that results from the mixing or combining of more than one active ingredient and includes both fixed and non-fixed combinations of the active ingredients. The term “fixed combination” means that the active ingredients are both administered to a patient simultaneously in the form of a single entity or dosage. The term “non-fixed combination” means that the active ingredients are both administered to a patient as separate entities either simultaneously, concurrently or sequentially with no specific time limits. The term “pharmaceutical combination” also applies the administration of three or more active ingredients.

The terms “treat” and “treatment” refer to therapeutic treatment, wherein the object is to prevent, reduce, relive or alleviate an undesired physiological symptom or disorder. Beneficial or desired clinical results include, but are not limited to, alleviation of symptoms, diminishment of extent of disease, stabilized (i.e., not worsening) state of disease, and amelioration the disease state.

The term “mammal” includes, but is not limited to, humans, mice, rats, guinea pigs, monkeys, dogs, cats, horses, cows, pigs, sheep, and poultry. The term “subject” or “patient” refers to a mammal, and in one embodiment, the patient is a human. The administering of the drug combination of the invention to the patient includes both self-administration and administration to the patient by another person.

The term “synergy” or “synergistic” as used herein refers to a therapeutic combination which is more effective than the additive effects of the two or more single active ingredients. Accordingly, synergic combination is meant that the therapeutic effect of the components of the combination is greater than the sum of the therapeutic effects of administration of any of these agents separately as a sole treatment. A synergistic effect may be attained when the active ingredients are: co-formulated and administered or delivered simultaneously in a combined, unit dosage formulation; or delivered by alternation or in parallel as separate formulations. When delivered in alternation therapy, a synergistic effect may be attained when the active ingredients are administered or delivered sequentially.

The term “potentiation” as used herein refers to a synergistic action in which the effect of the two or more single active ingredients delivered in combination is greater than the sum of the effects of each active ingredient delivered separately. This means that effect of one agent is enhanced by the other agent. Accordingly, the potentiation of the activity of a first active agent by a second agent in a combination therapy allows reducing the dose of the first agent administered to the subject, without affecting the beneficial clinical results obtained when using the standard/recommended dose.

The term “optional” or “optionally” as used herein means that a subsequently described event or circumstance may, but need not, occur, and that the description includes instances where the event or circumstance occurs and instances in which it does not.

The term “cardiac steroid” covers any member of the cardenolide and bufadienolide families.

The present invention relates to CSs and their use in a combination therapy with PI3K/Akt/mTOR inhibitors. Suitable CSs according to the invention are:
3-(alpha-L-Rhamnopyranosyloxy)-1beta,5beta,11alpha,14,19-pentahydroxy-5beta-card-20(22)-enolide (ouabain);
4-[(3 S,5R,8R,9S,10S,12R,13 S, 14S)-3-[(2S,4S,5R,6R)-5-[(2S,4S,5R,6R)-5-[(2S,4S,5R,6R)-4,5-dihydroxy-6-methyl-oxan-2-yl]oxy-4-hydroxy-6-methyl-oxan-2-yl]oxy-4-hydroxy-6-methyl-oxan-2-yl]oxy-12,14-dihydroxy-10,13-dimethyl-1,2,3,4,5,6,7,8,9,11,12,15,16,17-tetradecahydrocyclopenta[a]phenanthren-17-yl]-5H-furan-2-one (digoxin);
5-[(3 S,5R,8R,9S,10S,13R,14S,17R)-3,14-dihydroxy-10,13-dimethyl-1,2,3,4,5,6,7,8,9,11,12,15,16,17-tetradecahydrocyclopenta[a]phenanthren-17-yl]pyran-2-one (bufalin);
(3β,5β)-3-{[3-O-Acetyl-2,6-dideoxy-3-D-ribo-hexopyranosyl-(1->4)-2,6-dideoxy-β-D-ribo-hexopyranosyl-(1->4)-2,6-dideoxy-3-D-ribo-hexopyranosyl]oxy}-14-hydroxycard-20(22)-enolide (acetyldigitoxin);
(3β,5β,12β)-3-{[3-O-Acetyl-2,6-dideoxy-β-D-ribo-hexopyranosyl-(1->4)-2,6-dideoxy-β-D-ribo-hexopyranosyl-(1->4)-2,6-dideoxy-β-D-ribo-hexopyranosyl]oxy}-12,14-dihydroxycard-20(22)-enolide (acetyldigoxin);
5,14-dihydroxy-3-(5-hydroxy-4-methoxy-6-methyloxan-2-yl)oxy-13-methyl-17-(5-oxo-2H-furan-3-yl)-2,3,4,6,7,8,9,11,12,15,16,17-dodecahydro-1H-cyclopenta[a]phenanthrene-10-carbaldehyde (cymarin);
(3β,5β)-3-{[2,6-Dideoxy-β-D-ribo-hexopyranosyl-(1->4)-2,6-dideoxy-β-D-ribo-hexopyranosyl-(1->4)-2,6-dideoxy-β-D-ribo-hexopyranosyl]oxy}-14-hydroxycard-20(22)-enolide (digitoxin);
(3β,5β)-3,14-Dihydroxycard-20(22)-enolide (digitoxigenin);
(3β,5β,12β)-3,12,14-Trihydroxycard-20(22)-enolide (digoxigenin);
(3β,5β,12β)-3-{[2,6-Dideoxy-4-O-methyl-3-D-ribo-hexopyranosyl-(1->4)-2,6-dideoxy-β-D-ribo-hexopyranosyl-(1->4)-2,6-dideoxy-β-D-ribo-hexopyranosyl]oxy}-12,14-dihydroxycard-20(22)-enolide (metildigoxin);
(3S,5 S,8R,9S,10S,13R,14S,17R)-5,14-Dihydroxy-13-methyl-17-(5-oxo-2,5-dihydro-3-furanyl)-3-{[(2R,3R,4R,5R,6R)-4,5,6-trihydroxy-3-{[(2S,3R,4S,5 S,6R)-3,4,5-trihydroxy-6-(hydroxymethyl)tetrahydro-2H-pyran-2-yl]oxy}tetrahydro-2H-pyran-2-yl]oxy}hexadecahydro-1 OH-cyclopenta[a]phenanthrene-10-carbaldehyde (neoconvalloside);
(3β,5β)-3-{[2,6-Dideoxy-4-O-(3-D-glucopyranosyl)-3-O-methyl-β-D-ribo-hexopyranosyl]oxy}-5,14-dihydroxy-19-oxocard-20(22)-enolide (k-strophanthin);
(3β,5β)-3,5,14-Trihydroxy-19-oxocard-20(22)-enolide (k-strophanthidin);
5-[(5R,8R,9S,10S,13S,14S,17S)-10,13-dimethyl-2,3,4,5,6,7,8,9,11,12,14,15,16,17-tetradecahydro-1H-cyclopenta[a]phenanthren-17-yl]pyran-2-one (bufadienolide);
5-[(3 S,8R,9S,1 OR, 13R, 14S,17R)-14-Hydroxy-10,13-dimethyl-3-((2R,3R,4R,5R,6R)-3,4,5-trihydroxy-6-methyltetrahydro-2H-pyran-2-yloxy)-2,3,6,7,8,9,10,11,12,13,14,15,16,17-tetradecahydro-1H-cyclopenta[a]phenanthren-17-yl]-2H-pyran-2-one (proscillaridin);
(1β,3β,5β,11α)-1,3,5-(ethylidynetris(oxy)-11,14-dihydroxy-12,19-dioxobufa-20,22-dienolide (daigremontianin); and
(3β,5β,15β)-3,5-Dihydroxy-14,15-epoxybufa-20,22-dienolide (marinobufagenin).

According to a specific embodiment of the invention, the CS is ouabain, bufalin, or digoxin. According to a preferred embodiment, the CS is digoxin.

The pharmaceutical combinations of the present invention further include one or more PI3K/Akt/mTOR inhibitor. The term “PI3K/Akt/mTOR inhibitor” refers to an inhibitor of at least one component of this signal transduction pathway, i.e. the P13K, the Akt or the mTOR.

The inhibitor may be a small chemical molecule, an amino acid based molecule or a nucleic acid based molecule.

In accordance with one embodiment of the invention, the pharmaceutical combination includes an Akt inhibitor. This inhibitor may be an inhibitor of at least one of the isoforms of Akt, optionally at least two or more of the isoforms. Examples of Akt inhibitors suitable according to the invention, and their targets are provided in Table 1.

TABLE 1
List of inhibitors.

| Inhibitor Name | Akt | Akt1 | Akt2 | Akt3 | Other Targets |
|---|---|---|---|---|---|
| MK-2206 2HCl | | +++ | +++ | + | |
| Perifosine | + | | | | |
| (KRX-0401) | | | | | |
| GSK690693 | | ++++ | ++ | +++ | PKCθ, PKCη, PrkX |
| Ipatasertib | | ++++ | ++ | +++ | |
| (GDC-0068) | | | | | |
| AZD5363 | | ++++ | +++ | +++ | ROCK2 |
| PF-04691502 | ++++ | | | | PI3Kδ, PI3Kα, PI3Kγ |
| AT7867 | | ++ | ++ | ++ | PKA, p70 S6K |
| Triciribine | + | | | | HIV-1 |
| CCT128930 | | | +++ | | p70 S6K, PKA |
| A-674563 | | +++ | | | PKA, CDK2, GSK-3β |
| PHT-427 | + | | | | PDK-1 |
| Akti-1/2 | | ++ | + | + | |
| Afuresertib | | ++++ | ++++ | ++++ | |
| (GSK2110183) | | | | | |
| AT13148 | | ++ | + | ++ | PKA, ROCK2, ROCK1 |
| Miltefosine | ✓ | | | | PI3K, PKC |
| Honokiol | ✓ | | | | MEK |
| TIC10 Analogue | ✓ | | | | ERK |
| TIC10 | ✓ | | | | ERK |

Accordingly, the Akt inhibitor could be selected from:
MK-2206 2HCl (8-[4-(1-Aminocyclobutyl)phenyl]-9-phenyl[1,2,4]triazolo[3,4-f][1,6]naphthyridin-3(2H)-one dihydrochloride);
perifosine (1,1-dimethyl-4 [(octadecyloxy)hydroxyphosphinyl]oxy]-piperidinium inner salt, KRX-0401);
GSK690693 (4-[2-(4-amino-1,2,5-oxadiazol-3-yl)-1-ethyl-7-[[(3 S)-piperidin-3-yl]methoxy]imidazo[4,5-c]pyridin-4-yl]-2-methylbut-3-yn-2-ol);
ipatasertib ((2S)-2-(4-Chlorophenyl)-1-{4-[(5R,7R)-7-hydroxy-5-methyl-6,7-dihydro-5H-cyclopenta[d]pyrimidin-4-yl]-1-piperazinyl}-3-(isopropylamino)-1-propanone, GDC-0068);
AZD5363 (4-amino-N-[(1S)-1-(4-chlorophenyl)-3-hydroxypropyl]-1-(7H-pyrrolo[2,3-d]pyrimidin-4-yl)piperidine-4-carboxamide);
PF-04691502 (2-amino-8-[4-(2-hydroxyethoxy)cyclohexyl]-6-(6-methoxypyridin-3-yl)-4-methylpyrido[2,3-d]pyrimidin-7-one);
AT7867 (4-(4-chlorophenyl)-4-[4-(1H-pyrazol-4-yl)phenyl]piperidine);
Triciribine (5-Methyl-1-(β-D-ribofuranosyl)-1,5-dihydro-1,4,5,6,8-pentaazaacenaphthylen-3-amine);
CCT128930 (4-(4-Chlorobenzyl)-1-(7H-pyrrolo[2,3-d]pyrimidin-4-yl)-4-piperidinamine);
A-674563 ((2S)-1-[5-(3-methyl-2H-indazol-5-yl)pyridin-3-yl]oxy-3-phenylpropan-2-amine);
PHT-427 (4-dodecyl-N-(1,3,4-thiadiazol-2-yl)benzenesulfonamide);
Akti-1/2 (3-[1-[[4-(7-phenyl-3H-imidazo[4,5-g]quinoxalin-6-yl)phenyl]methyl]piperidin-4-yl]-1H-benzimidazol-2-one);
afuresertib (GSK2110183, N-[(2 S)-1-amino-3-(3-fluorophenyl)propan-2-yl]-5-chloro-4-(4-chloro-2-methylpyrazol-3-yl)thiophene-2-carboxamide);
AT13148 ((1S)-2-amino-1-(4-chlorophenyl)-1-[4-(1H-pyrazol-4-yl)phenyl]ethanol);
Miltefosine (hexadecyl 2-(trimethylazaniumyl)ethyl phosphate);
Honokiol (2-(4-hydroxy-3-prop-2-enylphenyl)-4-prop-2-enylphenol);
TIC10 Analogue (2,6,7,8,9,10-hexahydro-10-[(2-methylphenyl)methyl]-7-(phenylmethyl)-imidazo[1,2-a]pyrido[4,3-d]pyrimidin-5(3H)-one);
TIC10 (2,4,6,7,8,9-hexahydro-4-[(2-methylphenyl)methyl]-7-(phenylmethyl)-imidazo[1,2-a]pyrido[3,4-e]pyrimidin-5(1H)-one); and
MS-222 (ethyl-3-aminobenzoate methanesulfonate salt).

According to one embodiment of the invention, the Akt inhibitor is perifosine, which is an inhibitor belonging to a class of lipid-related compounds called alkylphospholipids. According to another embodiment, the Akt inhibitor is miltefosine (INN, trade names Impavido® and Miltex®), which is a broad-spectrum phospholipid antimicrobial drug. In accordance with a specific embodiment, the Akt inhibitor is ethyl-3-aminobenzoate methanesulfonate salt, designated hereinafter as “MS222” or “MS-222”.

It should be noted that the combination of the invention can also comprise more than two separate active ingredients as set forth above, i.e., three active ingredients or more. Accordingly, further to the two therapeutic agents specified above, the pharmaceutical combination of the invention can further comprise at least one additional therapeutic agent. A non-limiting example of the at least one additional therapeutic agent is an inotropic agent, such as a catecholamine, a beta blocker, a calcium blockers, and an ACE inhibitor.

The pharmaceutical combinations of the present invention are useful in treating or preventing a cardiovascular disease or disorder in a subject in need thereof. Thus, in one aspect, the present invention provides a pharmaceutical combination comprising a CS and at least one PI3K/Akt/mTOR inhibitor for use in treating a cardiovascular disease or disorder.

The term “cardiovascular disease or disorder” as used herein includes, but is not limited to, coronary heart disease, myocardial infarction, heart failure, chronic atrial fibrillation, acute atrial fibrillation, peripheral arterial disease, rheumatic heart disease, and congenital heart disease.

Heart failure is the condition in which cardiac output is not sufficient to meet the peripheral need for blood (i.e., oxygen). Usually reduction of below 50% of Cardiac Output (CO) is manifested by pathological conditions.

In another aspect, the present invention provides the use of a CS and at least one PI3K/Akt/mTOR inhibitor for the manufacture of a medicament for the treatment of a cardiovascular disease or disorder.

According to a different aspect, the invention provides a pharmaceutical combination of a CS and at least one PI3K/Akt/mTOR inhibitor for use in treating a cardiovascular disease or disorder.

According to a further aspect, the present invention provides a method of treating or preventing a cardiovascular disease or disorder by administering a CS and at least one PI3K/Akt/mTOR inhibitor to a subject in need thereof.

According to another aspect, the present invention provides a method for improving efficacy of the treatment of a cardiovascular disease or disorder with at least one PI3K/Akt/mTOR inhibitor comprising administering a combination comprising a CS and at least one PI3K/Akt/mTOR inhibitor to a subject in need thereof.

According to a still further aspect, the invention provides an inhibitor of the PI3K/Akt/mTOR cascade for use in potentiating the activity of a cardiac steroid.

According to another aspect, the invention provides a CS for use in a combination therapy with an inhibitor of the PI3K/Akt/mTOR cascade for treating a cardiovascular disease or disorder.

The activity potentiated by the inhibitor is selected from: (a) increase of the contraction force of the heart muscle; (b) regulation of heart rhythm; or (c) a combination of the (a) and (b).

According to a specific embodiment, the inhibitor potentiates the CS activity, thereby enabling to decrease of the amount of CS administered to a subject, while maintaining essentially the same clinical efficacy of the drug.

Thus, the inhibitor can be administered to a subject undergoing chronic or acute CS therapy, for reducing his dose of administered CS, while maintaining the clinical efficacy. The therapeutic efficacy can be measured by any acceptable means known in the art, for example, by an increase of left ventricular ejection fraction as measured by echocardiography, or by the amelioration of the symptoms of heart failure.

The beneficial combination allows reduction in the CS dose administered to a subject suffering from a cardiovascular disease or disorders, leading to a reduction in the side effects and enhancement of the long-term clinical effectivity of the CS in treatment.

The term “reduce” or “reduction” as used herein refers to any decrease in the dose of CS administered to a subject. For example, the dose of digoxin prescribed for chronic therapy in adults for maintenance ranges from about 3.4 to about 5.1 microgram/kg/day. The administration of a combination of digoxin and at least one Akt inhibitor according to the present invention allows the reduction of the CS dose to about 0.3-0.5 microgram/kg/day or less.

Digoxin is currently available as solutions and solid dosage forms (such as tablets) at various strengths, including 250 mcg/mL (0.25 mg/mL); 50 mcg/mL (0.05 mg/mL); 100 mcg/mL (0.1 mg/mL); 125 mcg (0.125 mg); 250 mcg (0.25 mg); 500 mcg (0.5 mg); 50 mcg (0.05 mg); 100 mcg (0.1 mg); 200 mcg (0.2 mg); 62.5 mcg (0.0625 mg); and 187.5 mcg (0.1875 mg). The advantageous combination of the invention enables to effectively treat cardiovascular diseases with a reduced daily dose of digoxin, such as 10% or less of the lowest amount specified above. Accordingly, in one embodiment, the daily dose of digoxin in the combination according to the invention may be about 5 mcg or less for tablets, or 5 mg/ml for solutions. For example, the effective amount of digoxin may be any one of 0.01, 0.1, 0.5, 1, 1.5, 2, 2.5, 3, 3.5, 4, 4.5, 5, 5.5, and 6 mcg per day of digoxin in the form of a tablet, or 0.01, 0.1, 0.5, 1, 1.5, 2, 2.5, 3, 3.5, 4, 4.5, 5, 5.5, and 6 mcg per day of digoxin in the form of a solution.

The CS dose reduction accessible by the administration of the PI3K/Akt/mTOR according to the present invention decreases the undesired side effects of the CS, while achieving the same beneficial clinical outcome as achieved with a higher dose. Undesired side effects of CS therapy include dizziness, fainting, changes in heart beat rate and arrhythmias, gastrointestinal and neurological symptoms, or a combination of two or more of the same. Accordingly, the present invention provides a combination for use in reducing side effects associated with standard CS therapy.

The present invention therefore particularly relates to additive and synergistic combinations of CS and PI3K/Akt/mTOR inhibitors, which are useful in treating subjects suffering from a cardiovascular disorder.

According to another embodiment, the active ingredients used by the invention or the composition comprising a combination thereof, may be administered via any mode of administration. For example, oral, intravenous, intramuscular, subcutaneous, intraperitoneal, parenteral, transdermal, intravaginal, intranasal, mucosal, sublingual, topical, rectal or subcutaneous administration, or any combination thereof.

According to a further embodiment, the active ingredients used by the invention; i.e. the CS and the PI3K/Akt/mTOR inhibitor, can be administered as separate unit dose forms. According to a still further embodiment, each of the active ingredients may be administered in a different administration mode. For example, when the CS (e.g., digoxin) is administered orally, the inhibitor can be delivered via a different route, such as intramuscularly, or when digoxin is administered intravenously (IV), the inhibitor is provided orally.

The currently acceptable treatment of atrial fibrillation includes administration of digoxin intravenously (IV), intramuscularly (IM) or orally (PO), under the following guidelines.

For rapid digitalizing (loading-dose) regimen:
IV: 8-12 mcg/kg (0.008-0.012 mg/kg) total loading dose; administer 50% initially; then may cautiously give ¼ the loading dose every 6-8 hours twice; perform careful assessment of clinical response and toxicity before each dose.
PO: 10-15 mcg/kg total loading dose (0.010-0.015 mg/kg); administer 50% initially; then may cautiously give ¼ the loading dose every 6-8 hours twice; perform careful assessment of clinical response and toxicity before each dose.

For maintenance regimen:
IV or IM: 0.1-0.4 mg every day. Notable, the IM route is not preferred due to severe injection site reaction.
PO: 3.4-5.1 mcg/kg/day or 0.125-0.5 mg/day. Dose may be increased every 2 weeks based on clinical response, serum drug levels, and toxicity.

The currently acceptable treatment of heart failure includes administration of digoxin according to the American College of Cardiology Foundation/American Heart Association (ACCF/AHA) guidelines. The loading dose to initiate digoxin therapy in patients with heart failure is not necessary between 0.125 to 0.25 mg PO/IV every day. Higher doses, including between 0.375 to 0.5 mg/day are rarely needed. It is recommended to use the lower end of dosing (0.125 mg/day) in patients with impaired renal function or low lean body mass.

In one aspect, the present invention provides a dosage form of digoxin comprising 5 mcg or less for use in treating a cardiovascular disease or disorder in combination with a PI3K/Akt/mTOR inhibitor.

The inhibitor in accordance with the invention is suitable for administration simultaneously, concurrently or sequentially in any order to the administration of the CS, e.g., digoxin. The supplementation of the CS therapy with the delivery of the Akt inhibitor may be used to lower the abovementioned administration doses of digoxin, thereby reducing the side effects associated with this drug. Importantly, the beneficial therapeutic effects of digoxin on the cardiovascular disorder (e.g., heart failure, atrial fibrillation) are maintained despite of the reduction in its administration dose.

It should be noted that the compositions of the invention may be administered in any conventional dosage formulation. Formulations typically comprise at least one active ingredient, as defined above, together with one or more acceptable carriers thereof.

The pharmaceutical compositions employed in the instant therapy can be administered in various oral forms including, but not limited to, tablets, capsules, pills, powders, granules, elixirs, tinctures, suspensions, syrups, and emulsions. It is contemplated that the active ingredients can be delivered by any pharmaceutically acceptable route and in any pharmaceutically acceptable dosage form. These include, but are not limited to the use of oral conventional rapid-release, time controlled-release, and delayed-release pharmaceutical dosage forms. The active drug components can be administered in a mixture with suitable pharmaceutical diluents, excipients or carriers (collectively referred to herein as “carrier” materials) suitably selected to with respect to the intended form of administration.

More particularly, since the present invention relates to the treatment of diseases and disorders with a combination of active ingredients which may be administered separately, the invention also relates, as a further aspect, to combining separate pharmaceutical compositions in a kit form. Accordingly, by another aspect, the present invention concerns an article of manufacture in the form of a kit comprising unit dosage forms of cardiac steroid and at least one separate unit dosage form of a PI3K/Akt/mTOR inhibitor as described above. In one embodiment, the CS is digoxin.

The kit may further comprise a label or package insert, which refer to instructions customarily included in commercial packages of therapeutic products, that contain information about the indications, usage, dosage, administration, contraindications and/or warnings concerning the use of such therapeutic products. The kit may further comprise directions for the simultaneous, sequential or separate administration of the first and second pharmaceutical compositions to a subject.

According to one embodiment, the kit may comprise (a) a first container with a CS (b) a second container with PI3K/Akt/mTOR inhibitor; and optionally (c) a third container with a third pharmaceutical formulation, wherein the third pharmaceutical formulation comprises an additional active ingredient. A non-limiting example of the additional active ingredient is an inotropic agent, such as a catecholamine, a beta blocker, a calcium blocker, and an ACE inhibitor.

The kit form is particularly advantageous when the separate components (i.e., the active ingredients) are administered in different dosage forms (e.g., oral and parenteral), or are administered at different dosage intervals.

It should be appreciated that both components of the kit, the CS in the first dosage form and the inhibitor in the second dosage form, may be administered simultaneously. Alternatively, the CS dosage form and the inhibitor are administered sequentially in any order.

In one embodiment, both the CS and the inhibitor in the kit are adapted for oral administration, and may be packaged as two different oral dosage forms (e.g., pills, capsules). According to another embodiment, the CS and the inhibitor of the kit are provided as two different dosage forms, for example, digoxin PO and inhibitor IV, or vice versa. According to a specific embodiment, both the CS and the inhibitor are administered IV. In such case, the kit may comprise two separate vials of each of the active agents (in a suitable carrier).

According to one embodiment, the kit of the invention is intended for treating cardiovascular disease or disorder, such as coronary heart disease, myocardial infarction, heart failure, chronic atrial fibrillation, acute atrial fibrillation, peripheral arterial disease, rheumatic heart disease, or congenital heart disease. As indicated above, the treatment is intended for achieving the same therapeutic effect in a subject suffering from a cardiovascular disease or disorder as that achieved by CS administered alone. The therapeutic effect is maintained while reducing the administration dose of CS, compared to the amount administered without the inhibitor, hence resulting in the reduction of the undesired side effects associated with CS therapy.

In another aspect, the present invention provides a pharmaceutical composition comprising a combination of CS and a PI3K/Akt/mTOR inhibitor. The active ingredients of the present invention are generally administered in the form of a pharmaceutical composition comprising both a CS and an inhibitor as defined above together with a pharmaceutically acceptable carrier or diluent, and optionally a further therapeutic agent. Thus, the active ingredients used by this invention can be administered either individually in a kit or together in any conventional oral, parenteral or transdermal dosage form.

It should be noted that the combination of the invention is use for treating the same indications as the CSs, such as heart failure and atrial fibrillation. Importantly, the dosage of the CS in the combination is lower than the dosage of the CS administered alone for the same indication and for the same mode of administration.

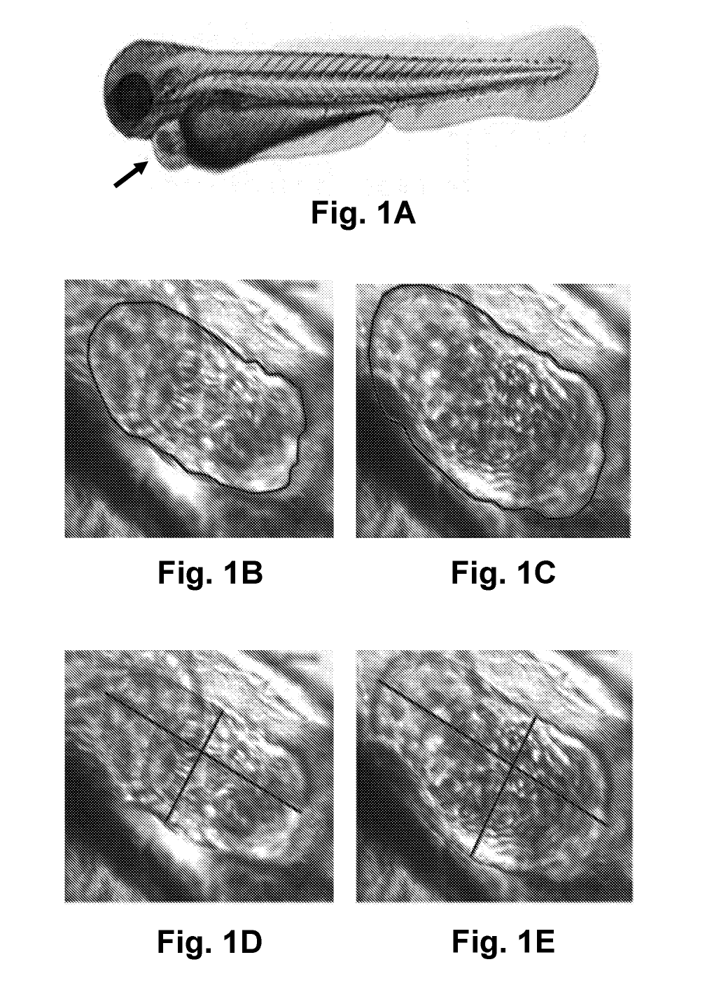

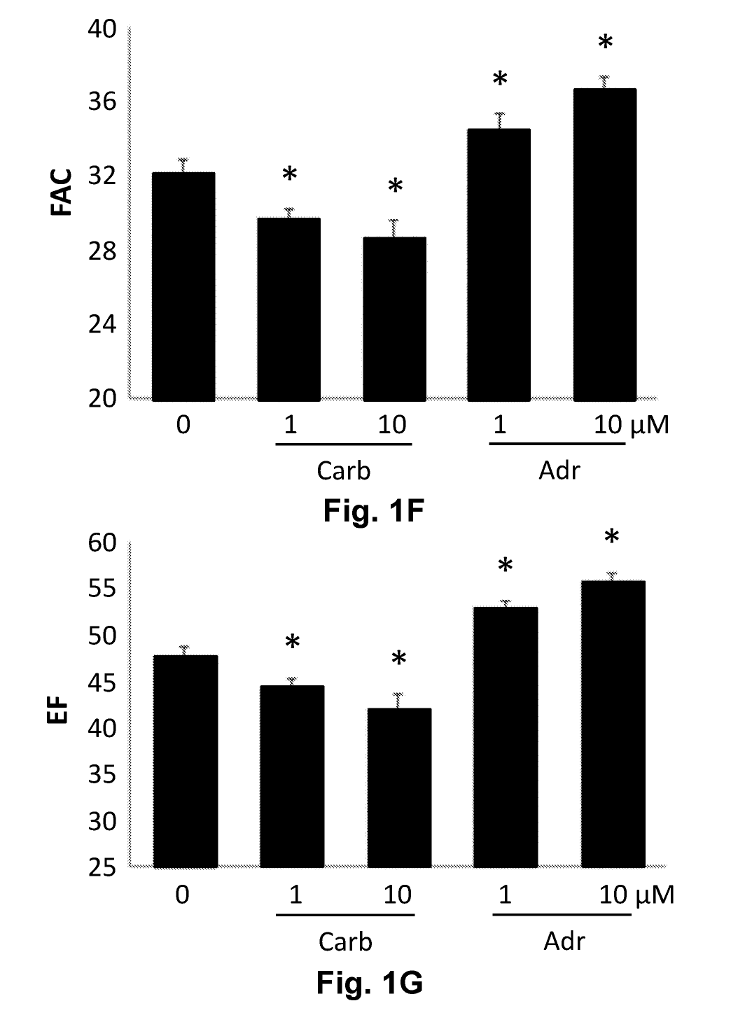

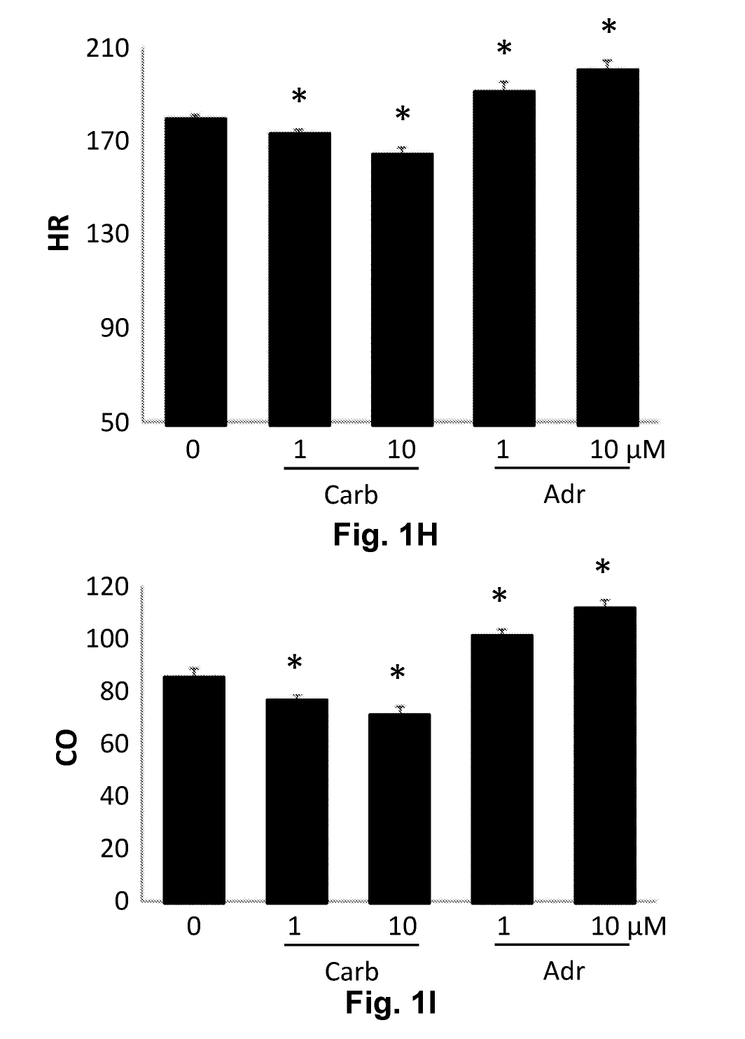

The Examples provided herein demonstrate the potentiating, additive and/or synergistic effects of the combinations of the invention in both zebrafish and rat models. The zebrafish is a well-established experimental model for the study of embryonic development. In recent years it has entered the field of cardiovascular research as an organism offering distinct advantages for dissecting molecular pathways of cardiovascular development, regeneration and function. In the present invention zebrafish larvae were used to address the hypothesis that Akt signaling pathways play a role in CS-induced increases in heart contractility. To address this issue, an in-vivo cardiac function system for zebrafish larvae was established. The effects of CS and kinases inhibitors were studied using live imaging of the fully developed cardiovascular zebrafish larvae. The method is based on the assumption that the heart ventricle has an elliptic shape and its contraction changes the area between systole and diastole. In the experiments presented in the Examples, the measurements were improved by determinations of the ventricular area using continuous drawings of the polygon border of the ventricle, yielding accurate FAC and EF of the heart in-vivo (FIGS. 1B-1E). The system was validated by testing the effects of adrenergic and cholinergic agonists added to the swimming media on zebrafish heart contractility. As expected, adrenalin (1 μM) induced a significant increase in heart contractility and rate (FIGS. 1F-1I). This classical response resembles that seen in other species. Similarly, cholinergic agonists (i.e. acetylcholine, 1 μM) induced a decrease in heart contractility and rate (FIGS. 1F-1I) comparable to its effects in other animal models. Hence, this method of cardiac measurement enables efficient determination of key aspects of cardiac function, such as FAC, EF, heart rate and calculation of CO of zebrafish larvae, and can be used for in-vivo physiological and pharmacological investigations.

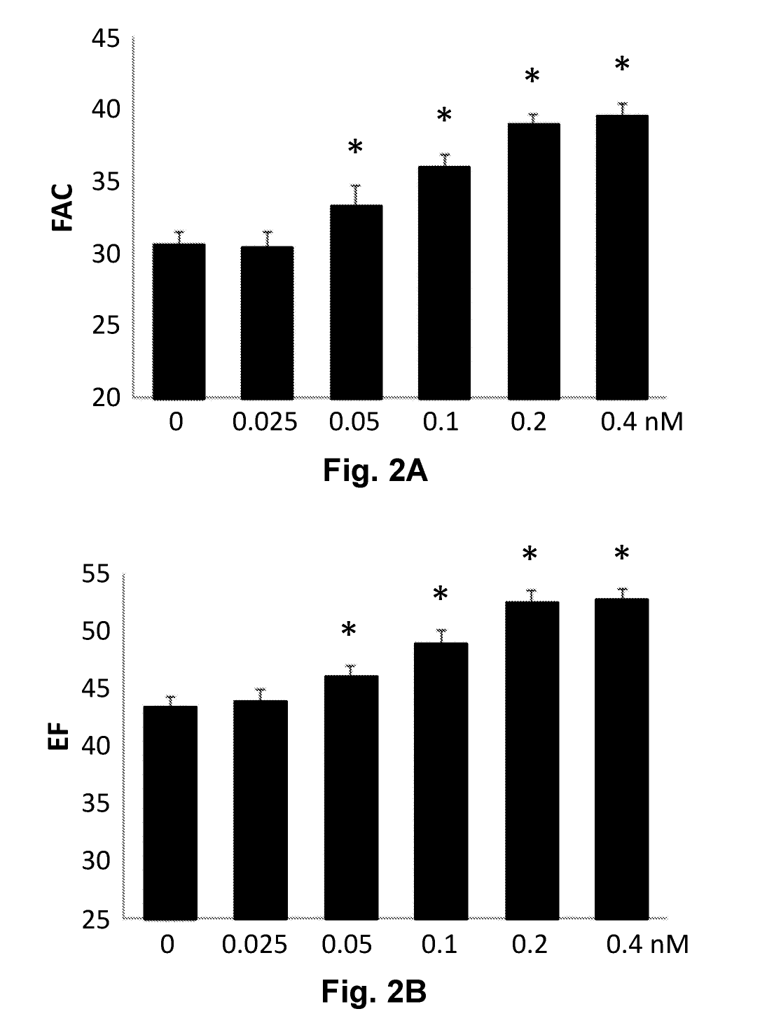

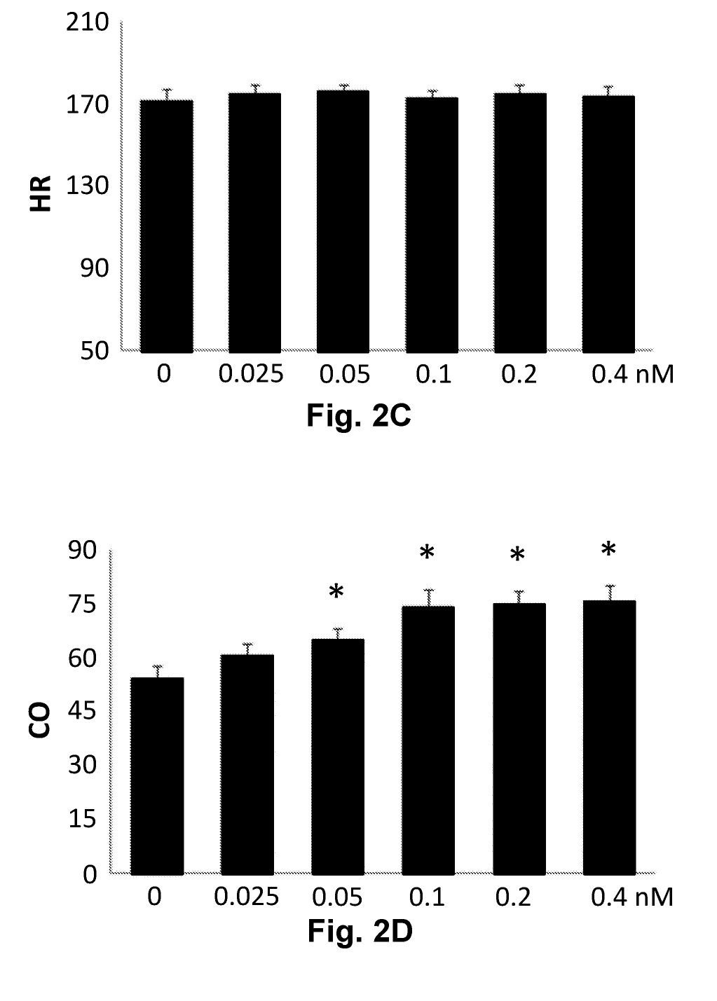

The addition of ouabain, digoxin or bufalin to zebrafish larvae swimming medium, increased, dose-dependently, the force of contraction of heart muscle. Whereas 0.1 nM bufalin or ouabain produced a significant increase in heart contractility, an about tenfold higher concentration of digoxin was required to yield a similar effect (FIGS. 2-4). This effect and the differential potencies are similar to the one seen in mammals, demonstrating the suitability of the zebrafish model for the study of CS action. CS-induced toxicity, manifested by reduced FAC, EF or mortality, was observed at concentrations 1000 times higher than those required for a significant positive inotropic effect, indicating a wider dose-response for CS in this organism.

Inhibition of Na⁺, K⁺-ATPase by CS is the recognized mechanism of action for their ability to increase the force of contraction of heart muscle. The exclusiveness of this mechanism was challenged by the demonstration that the interaction of CS with Na⁺, K⁺-ATPase elicits the activation of several major signaling cascades, including extracellular signal-regulated kinases (ERK), Akt, and iNOS. These signaling pathways, once activated by CS, participate in numerous physiological functions including cell viability, kidney and muscle function and cardiac hypertrophy. Indeed, using an ex-vivo experimental system, it was shown that inhibition of Src tyrosine kinase (Src) and ERK attenuated the CS-induced increase in heart contractility by affecting Ca⁺² homeostasis.

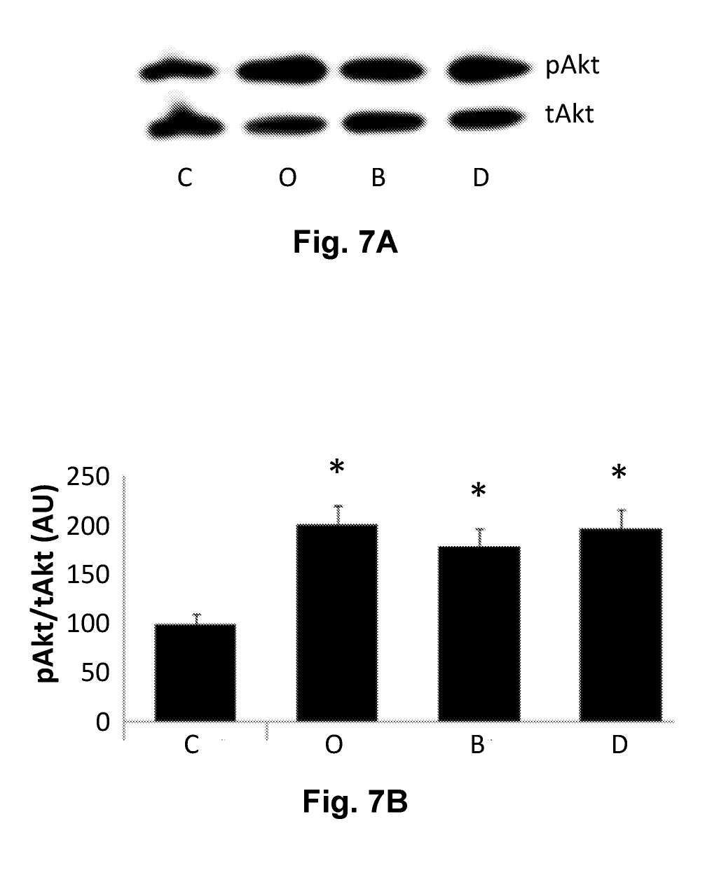

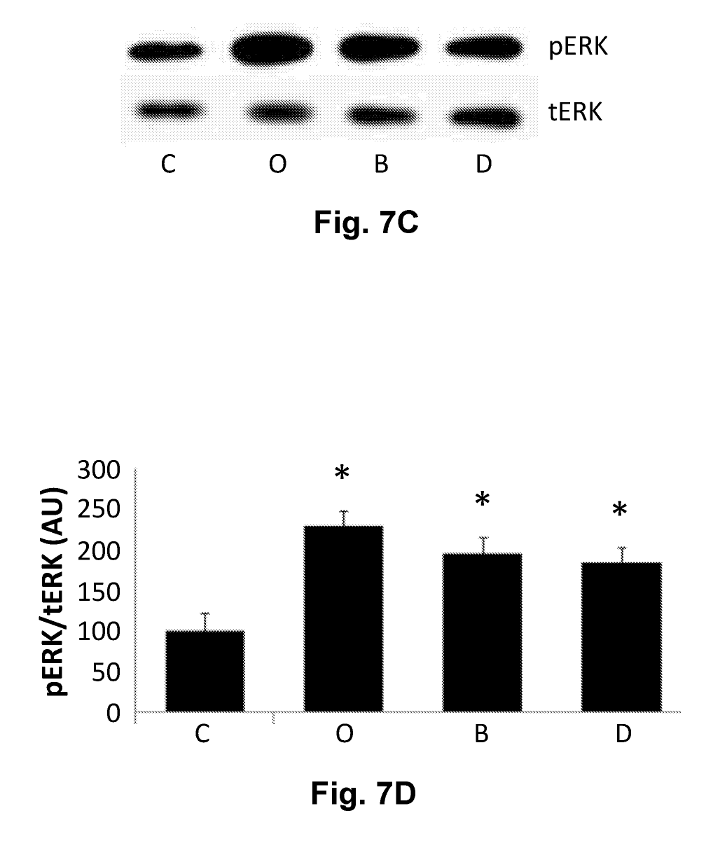

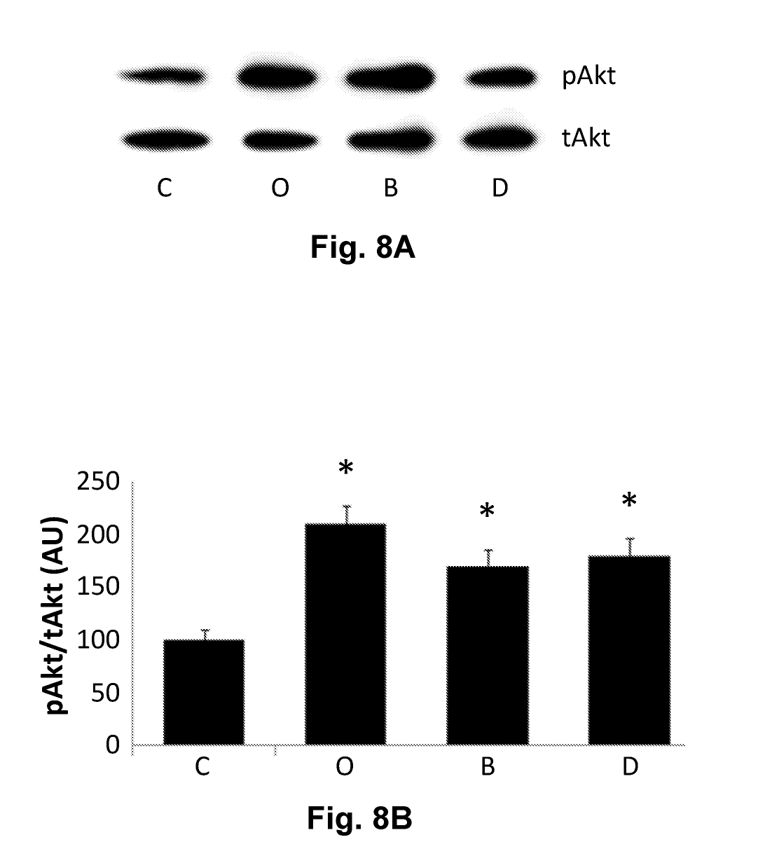

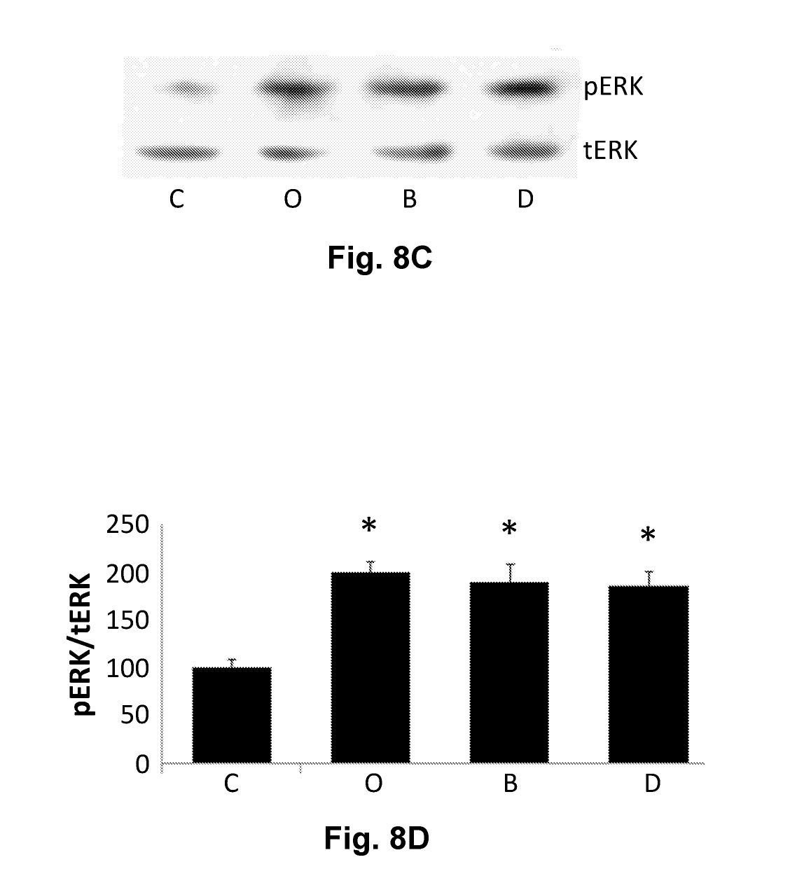

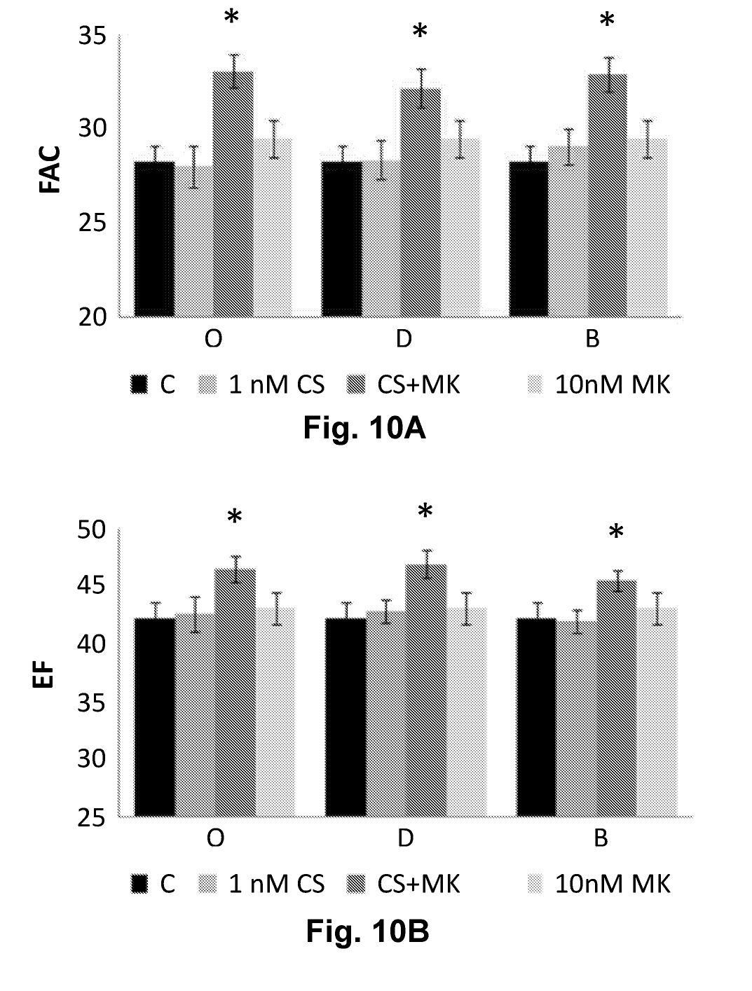

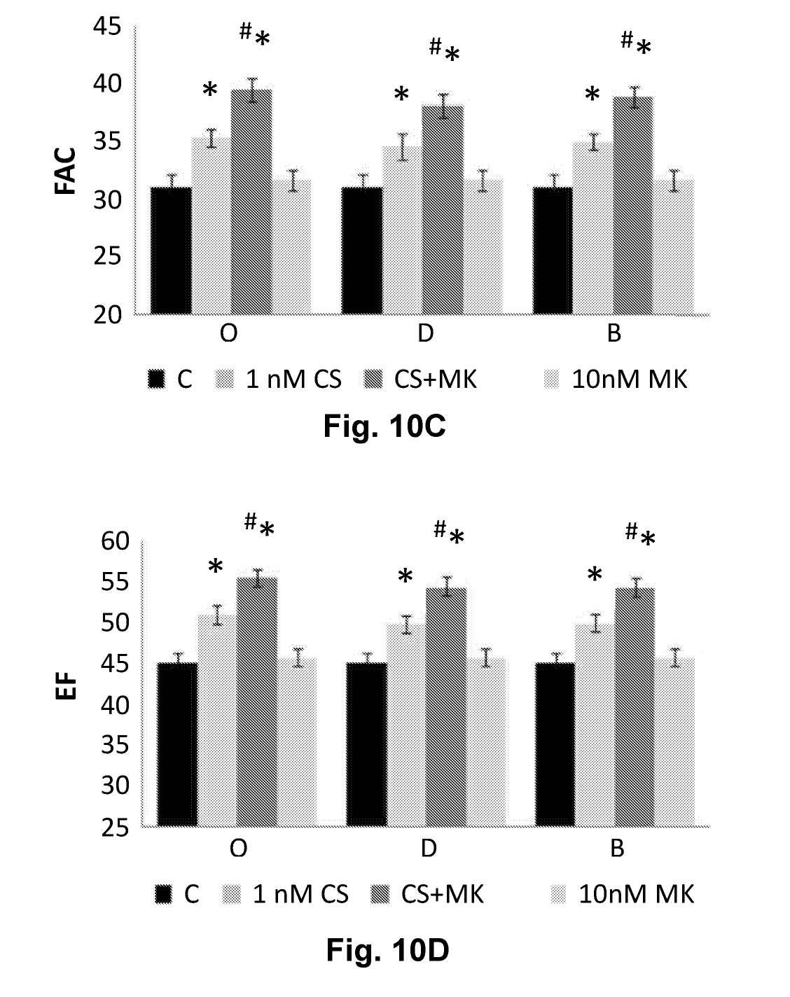

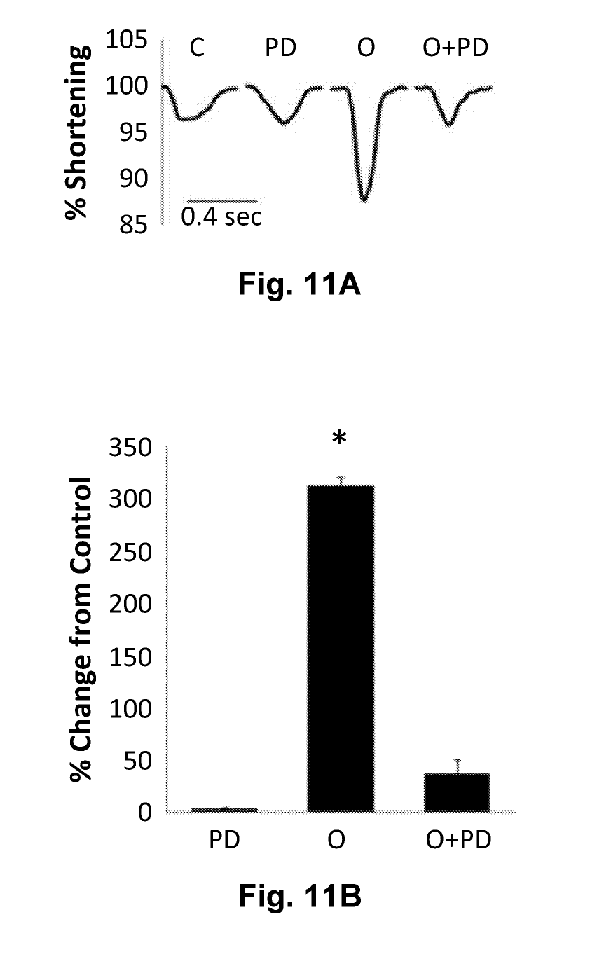

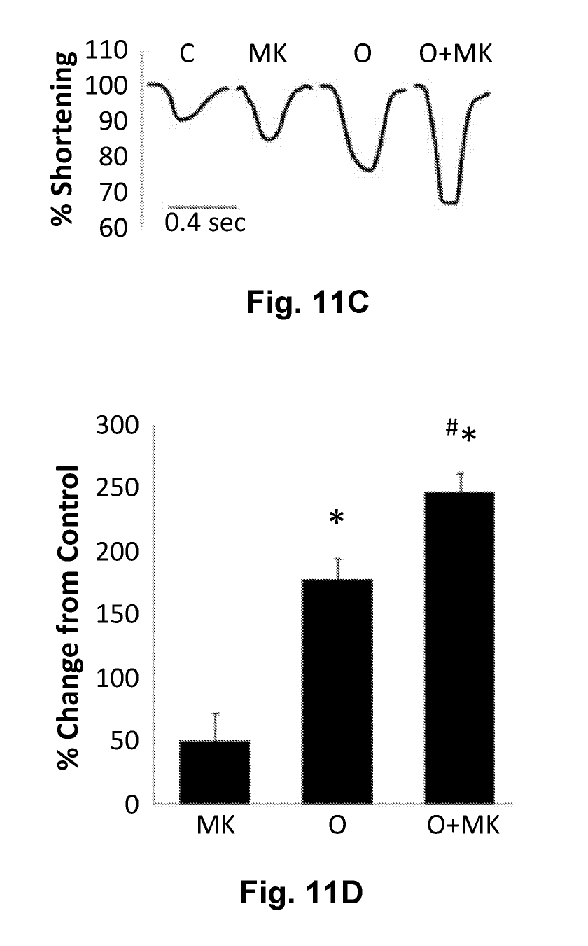

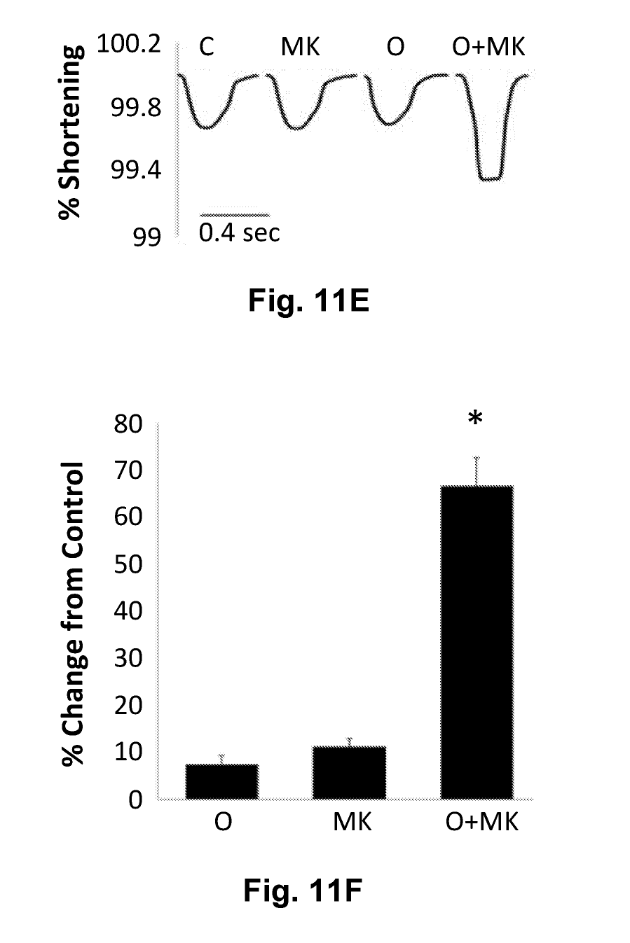

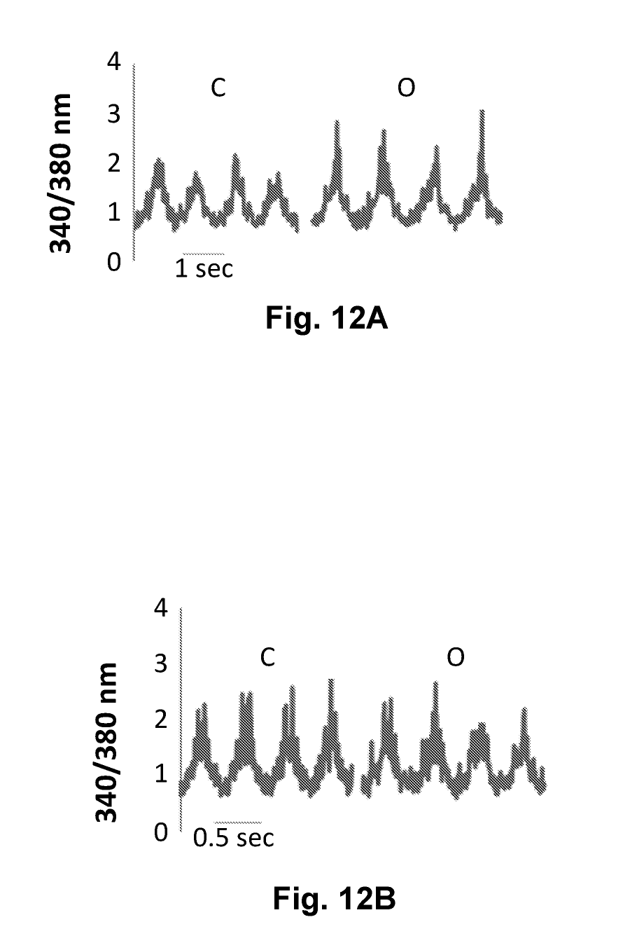

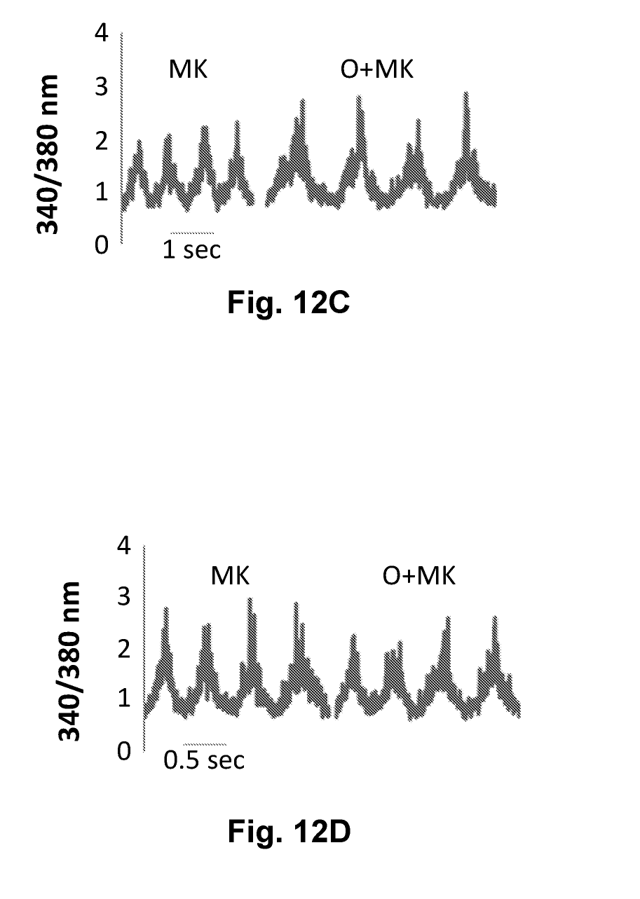

The effect of CS and Akt phosphorylation was shown in many tissue culture cells including rat brain and heart, and opossum kidney proximal tubular cells. Similarly, the exposure of adult zebrafish to CS caused an about twofold increase in ERK and Akt phosphorylation in heart tissue in the wild-type (wt) fish (FIG. 7) and in the Zebrafish Accordion (acc) mutants (FIG. 8). The protein phosphorylation may result in conformational changes following the interaction of CS with the Na⁺, K⁺-ATPase. Alternatively, the phosphorylation may be the consequence of indirect mechanisms, such as changes in intracellular Ca⁺² or in muscle tension. The observation that CS did not increase the heart force of contraction nor Ca²⁺ transients but did augment ERK and Akt phosphorylation in the acc mutants favors the first mechanism. The inventors discovered that the addition of Akt inhibitor in-vivo and ex-vivo potentiated the CS-induced increase in contractility (FIGS. 10C-10D). Furthermore, the exposure of the acc mutants to the Akt inhibitor caused the appearance of CS-induced increase in heart contractility at concentrations that were ineffective in its absence (FIGS. 10A-10B and FIGS. 11E-11F). Measurements of changes in CS-induced Ca²⁺ transients in zebrafish heart (FIG. 12, Table 3) revealed that Akt inhibition did not affect the ouabain-induced increase in Ca²⁺ transient amplitude in wt. Similarly, in acc mutant, where CS induced increase in force of contraction in the presence of Akt inhibitor, CS did not cause an increase in Ca²⁺ amplitude. As inhibition of Akt in both the wt and in acc mutants resulted in a similar phenotype, without concomitant changes in Ca²⁺ transients, the inventors concluded that the mechanisms involved in this effect are not associated with alterations in intracellular Ca²⁺ and may be a consequence of other changes such as gene expression or Ca²⁺ sensitivity.

Taking into consideration all the findings, it appears that CS-induced increases in heart contractility result from two pathways. Inhibition of Na⁺, K⁺-ATPase following CS binding increases the concentration of intracellular Na⁺ and, consequently, the cytoplasmic Ca⁺² level and contractility. Concurrently, CS binding to Na⁺, K⁺-ATPase activates intracellular signaling pathways that regulate contractility: CS-induced ERK phosphorylation, presumably directly or by increasing Ca⁺² turnover, augments the CS-induced effect. On the other hand, CS-induced Akt phosphorylation, by unknown mechanisms which may involve changes in the contractile machinery and/or reduced Ca⁺² sensitivity, attenuates the CS-induced effects. The final outcome of the activation of the signaling pathways emerges from the balance between the two branches. This concept of CS-induced effects suggests that changes in ERK and Akt activities, by inhibitors or genetic manipulation, have a significant influence on the efficacy and toxicity of the CS.

The invention will now be described with reference to specific examples and materials. The following examples are representative of techniques employed by the inventors in carrying out aspects of the present invention. It should be appreciated that while these techniques are exemplary of specific embodiments for the practice of the invention, those of skill in the art, in light of the present disclosure, will recognize that numerous modifications can be made without departing from the spirit and intended scope of the invention.

### EXAMPLES

### Materials and Methods

### Aquaculture

Experiments were performed on wild-type (WT, AB strain) zebrafish (Danio rerio). The fish were maintained in accordance with the principles established by the NIH. The Hebrew University Animal Care Committee approved the use of the animals and the experimental protocols used in this study (Approval #MD-11-12979-2). The fish were kept in swimming medium (E3 medium) in small aquaria at 28° C. and maintained under a 14h: 10h light:dark cycle. The zebrafish larvae were maintained in the presence of 0.1% methylene blue (M9140, Sigma-Aldrich Inc, Israel) at 28° C. All measurements were taken in embryos at 72 hours post fertilization (hpf).

### Pharmacological Manipulations

Ouabain, bufalin, digoxin, acetylcholine, carbachol, protein phosphatase 2 (pp2) and U0126 (1,4-diamino-2,3-dicyano-1,4-bis(2-aminophenylthio)butadiene) were purchased from Sigma-Aldrich Inc., Israel. The compounds were dissolved in egg water (0.3 g Instant Ocean Salt in distilled water) to a final selected concentration, as specified in the following examples. The larvae in 20 μL of E3 medium were transferred to the egg water solution containing one or two of the above specified drugs of choice, and incubated for 60-90 minutes at 28° C. At selected time intervals, as specified in the following examples, at 28° C., the larvae were transferred to filming/anesthetizing medium, comprising 0.1% SeaKem LE Agarose (BMA, Rockland, Me., USA) and 15 μM MS222 (ethyl-3-aminobenzoate methanesulfonate salt (Sigma-Aldrich Inc., Israel).

Filming process Zebrafish larvae heart imaging was performed using an Olympus CKX41 (Japan) upright microscope with ×10 or ×20 magnification, and integrated incandescent illumination. A FastCam imi-tech (Korea) high speed digital camera with 640×480 pixel gray scale image sensor was mounted on the microscope, using ImCam software (IMI Technology, Co. Ltd) for high speed video recording. The larvae were anesthetized by placing them in filming/anesthetizing medium. Each larva in 0.5 ml filming/anesthetizing medium was transferred to a 96-well tissue culture plate at room temperature and sequential images of the heart were obtained, with the larvae positioned on their side, at 80 fps during 10 seconds with a shutter speed of 0.016 seconds.

### Quantification of Heart Contractility

Physiological parameters of cardiovascular performance, as indicated hereinbelow, were evaluated in the zebrafish larvae. Image analysis applications ImageJ (NIH, USA) were used, allowing the delineation of the endomyocardial border at the end of systole or end of diastole to define the ventricular area. Sequential still frames were analyzed to capture ventricular end-systole and end-diastole images. End Systolic Area (ESA, FIG. 1B) and End Diastolic Area (EDA, FIG. 1C) were used to calculate Fractional Area Change (FAC), according to the equation: FAC=(EDA-ESA)/EDA*100. The Ejection Fraction (EF) was determined by an independent estimate of ventricular volume. This was achieved by placing scan lines across the midventricular short axis (SA) and long axis (LA) at the end of systole (FIG. 1D) and diastole (FIG. 1E). These parameters were used to quantify the End Systolic volume (ESV) and End Diastolic Volume (EDV) using the volume equation for an ellipsoid: Vol=4/3*3.14*LA*SA². EF was then calculated according to the equation: EF=(EDV-ESV)/EDV*100. Heart rate (HR) was measured in beats per minute. Cardiac output (CO) was calculated according to the equation: CO=(EDV-ESV)*HR. A minimum of five sequential pairs of systolic and diastolic cycles were measured and analyzed in 9-12 larvae for each treatment. Each of the presented experiments was repeated at least 3 times, with identical results.

### Measurements of Ca²⁺ Transients

Spontaneous Ca²⁺ transients were measured in isolated hearts from adult zebrafish. The zebrafish were stunned by a blow to the head and the hearts were removed quickly and placed in Dulbecco's Modified Eagle's Medium (DMEM) containing 10% Fetal Bovine Serum (FBS) at room temperature. A total 3-5 hearts were placed in a small Petri dish containing 100 μl Krebs-Ringer's solution (119 mM NaCl, 2.5 mM KCl, 1 mM NaH₂PO₄, 2.5 mM CaCl₂, 1.3 mM MgCl₂, 20 mM HEPES and 11 mM D-glucose) with 0.01 mM Fura-2 AM (Biotium, Inc., CA, USA) in DMEM-FBS. Following incubation for 10 min (37° C., 5% CO₂), 150 μl of Krebs-Ringer's solution were added to the Petri dish which was incubated for an additional 30 minutes. The dye was removed from the solution by two incubations (10 minutes each, room temperature) in DMEM-FBS solution. Intracellular Ca²⁺ transients were measured in stabilizing medium containing 1% low-melt agarose in Krebs-Ringer's solution. The hearts were kept for 30 minutes in the presence of MK-2206 (or saline) and then underwent excitation/emission cycles for 2 minutes at 340 and 380 nm with 510 nm emission using a PTI fluorimetric system (Photon Technology International, Madison, USA). Ouabain was then added and the excitation/emission cycles were monitored for 8 minutes. The Ca²⁺ levels are presented as the ratio of 340/380 nm fluorescence emission.

### Isolation of Zebrafish Ventricular Myocytes

Adult (4 to 12-month-old) ventricular myocytes were obtained by enzymatic dissociation (with collagenase II and IV, at 5 mg/ml, each). The zebrafish were stunned by a blow to the head and the brain was pithed. The heart was quickly removed and placed in a small Petri dish containing 10 ml isolation solution: 100 mM NaCl, 10 mM KCl, 1.2 mM KH₂PO₄, 4 mM MgSO₄, 50 mM taurine, 20 mM glucose, and 10 mM Hepes, pH 6.9. The ventricle was cut free from the bulbus and atrium under a binocular. Ventricles from 3 fish were incubated for 45 minutes at 32° C. in a solution containing perfusion buffer (150 mM NaCl, 5.4 mM KCl, 1.5 mM MgSO₄, 0.4 mM NaH₂PO₄, 2 mM CaCl₂, 10 mM glucose, and 10 mM Hepes, pH 7.7). Collagenases II and IV (Gibco, NY, USA, 5 mg/ml each) and additional CaCl₂ (2.012 mM final concentration) were added. Following Eppendorf centrifugation (1 minute, 250×g at room temperature) the precipitated cells were suspended in 1 ml perfusion buffer for 30 minutes at room temperature before use. Spontaneous contraction was observed in about 10% of the cells in the preparation.

### Measurement of Cardiomyocyte Contractility

Cells were transferred to a chamber with a quartz base and examined using an inverted epifluorescence microscope (Nikon Diaphot 200, Japan). The myocytes were field-stimulated (0.5 Hz, 70 V, square waves) and contractions were measured using a video motion edge detector (Crescent Electronics, Sandy, Utah, USA) at the rate of 5 Hz. Cardiomyocyte performance was calculated as the percentage of resting cell length. The slopes of contraction and relaxation (+dL/dt and −dL/dt) were calculated from the linear portions of the changes in contractility. 9 cells per each group were measured. Each of the experiments was repeated at least 3 times with identical results.

Heart Dissection and Protein Extraction from Adult Zebrafish

Adult zebrafish (˜12-months-old) were transferred to swimming medium containing 1 μM of CS drugs, as specified in the following examples. At various time points (5-30 min), as indicated in the following examples, the zebrafish were transferred to perfusion buffer and the hearts were immediately removed and transferred to RIPA lysis buffer (Sigma-Aldrich), and protease inhibitor cocktail (P8340, Sigma-Aldrich), at a 1:100 dilution). The tissue was homogenized in an ultrasonic homogenizer (Microson, NY, USA) and aliquots of the homogenate were stored at −70° C. until used.

### Western Blotting

Protein dilution and separation on SDS-PAGE electrophoresis, their transfer to a polyvinylidene fluoride membrane were carried out. The membranes were incubated for 1 hour at room temperature with one of the specific antibodies against Phospho-p44/42 MAPK (Erk1/2) (Thr202/Tyr204), Rabbit mAb #4370 (Cell signaling) or Phospho-Akt (Ser473) (193H12), Rabbit mAb #4058 (Cell signaling), at a 1:1000 dilution in TBS containing 0.1% Tween. The membranes were washed with TBS containing 0.1% Tween and exposed to horseradish peroxidase-conjugated secondary goat anti-rabbit IgG antibody (1:50,000). The membranes were stripped prior to their exposure to a different antibody. Detection was carried out with the aid of a Luminata™ Crescendo Western HRP Substrates, according to the manufacturer's instructions. Preliminary experiments verified that the stripping and re-blotting procedure did not affect the quantification of any of the proteins.

### Rat Model

Male Wister rats (150-175 g body weight) were used. Left anterior descending artery (LAD)-ligation was performed in all animals. The rats were divided to four groups, each treated with subcutaneous injection with saline (0.5 ml/kg/day), ouabain (0.8-8 mg/kg/day), MK-2206 (12 mg/Kg/day) or a combination of MK-2206 and ouabain. All injected solutions contained 9% DMSO. Heart contractility in-vivo was monitored using echocardiography. At the end of the treatment period, animals were sacrificed and heart and blood samples were harvested for analysis. Blood samples were also collected (from the eye) at baseline and 24 hours post myocardial infarction (MI).

### LAD-Ligation

Rats were anesthetized with 10% ketamine, 2% xylazine (0.1 ml/kg) and ventilated with a small animal respirator (Harvard). The heart was exposed via left sternotomy, and the LAD-ligation was induced by placing a 6-0 silk suture around the left anterior descending coronary artery near the atrial auricle. The animals were allowed to recover and treated with 0.5 mg/100 g Tramadol Hydrochloride by subcutaneous injection for post-operative analgesia.

### Rat Echocardiography

Echocardiograms were performed on rats anesthetized with 2% isoflurane at baseline, at 24 hours, as well as at 3, 6 and 10 days post LAD-ligation, using a VEVO 770 equipped with a 30 MHz linear transthoracic transducer (VisualSonics). Measurements were performed in triplicate using the leading-edge convention for myocardial borders as defined by the American Society of Cardiology. Left ventricular, end-diastolic and end-systolic diameters (LVEDD and LVESD), diastolic and systolic interventricular septal diameters (IVSD and IVSS), left ventricular diastolic area (LVEDA), left ventricular systolic area (LVESA), and heart rate (HR) were measured. Shortening fraction (SF) was calculated by the equation: SF (%)=(LVEDD−LVESD)/LVEDD×100. Heart rate was measure by peak intervals of the echocardiography measurements. All measurements were performed by an individual who was blind to the entire experimental protocol and animal treatments.

### Masson's Trichrome Staining

Masson's Trichrome staining was used to detect fibrosis of Left ventricle (LV) myocardium. Heart muscles were placed in 4% paraformaldehyde for 72 hours. Paraffin embedded sections of 5 μm thickness were performed from the ligation area to the apex. Tissue sections were deparaffinized, rehydrated with graded ethanol, immersed in Bouin's solution and incubated overnight at room temperature. Following wash with running tap water for 5 minutes, the heart sections (Nuclei) were stained with Weigert Hematoxylin for 5 minutes, washed in running tap water for 5 minutes and rinsed with deionized water for 5 minutes. Heart muscle was stained red by incubation with Biebrich Scarlet-Acid-Fuschin (Sigma-Aldrich) for 5 minutes, following rinse with deionized water for 5 minutes and immersion in phosphomolybdic phosphotungstic acid (Sigma-Aldrich) for 5 minutes. Subsequently, collagen was stained blue by incubation in Aniline Blue (Sigma-Aldrich) for 5 minutes and rinsed in 2% acetic acid for 2 minutes. Finally, the tissues were rehydrated with ethanol, and mounted with VectaMount (VECTOR, California, US). The slides were viewed in Nikon TL microscope (×20), photographed, and the total section area of the myocardium in the tissue sections was measured using ImageJ software. Three selected sections were quantified for each animal. After determining the area of each heart and the fibrosis region, the relative area of the fibrotic tissues was calculated.

### Plasma Measurements

Blood samples were collected from rat eye at baseline and at 24 hrs. Post LAD-ligation. The plasmas of 11 animals were tested for the indicated biomarkers of MI.

### Statistics

A mixed within/between Repeated Measures Analysis of Variance was performed using the SPSS program. Repeated measures were the five length measurements of each cell. Between group measures were the four experimental groups (control, kinase inhibitor, CS, and both). None of the within-subject effects were significant, indicating that the five length measurements of each cell were not significantly different from each other neither when collapsed across the experimental groups F(4,27)=0.273, p=0.936) nor within the experimental groups (F(12, 87)=0.902, p=0.548), supporting the stability of the measurements for each cell. The between-subject was significant (F(3,30)=16.047, p=0.000). Post-hoc between group differences were analyzed using Bonferroni-adjusted Student's t-test. In all other experiments using the zebrafish model, difference between groups was assessed using Student's t-test. For experiments on rat model, data were expressed as the Mean+SEM. Two-tailed t test was applied and P<0.05 was considered statistically significant.

### Example 1

### Validation of Heart Contractility Measurements in Zebrafish Larvae

To validate the in-vivo heart contractility measurements, the effects of adrenergic and cholinergic agonists on the heart contractility of wt zebrafish larvae were tested. As shown in FIG. 1, treatment of zebrafish larvae with carbachol decreased the heart force of contraction and rate (FIGS. 1F-1H), resulting in a reduction of 10.39±2.2% and 16.92±3% in cardiac output (CO) (FIG. 1I) at 1 and 10 μM, respectively. Similar results were obtained with acetylcholine. Larvae exposed to adrenalin showed the opposite effect, i.e. an increase in heart contractility and rate, resulting in an increase of 18.34±1.97% and 30.48±2.67% in CO at 1 and 10 μM, respectively (FIG. 1I). Similar results were obtained with noradrenalin. These results are in complete agreement with the well-established effects of these compounds, demonstrating the capability of the zebrafish experimental system to identify changes in heart contractility and rate under physiological conditions.

### Example 2

### CS-Induced Increase in Heart Contractility in Zebrafish Larvae

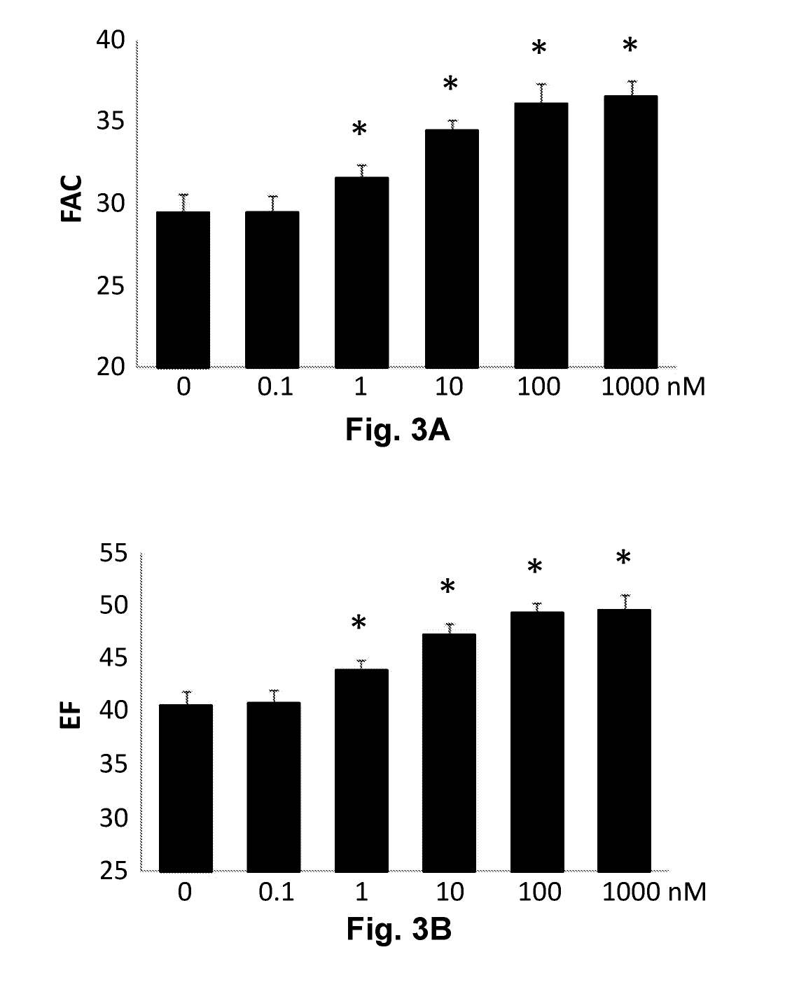

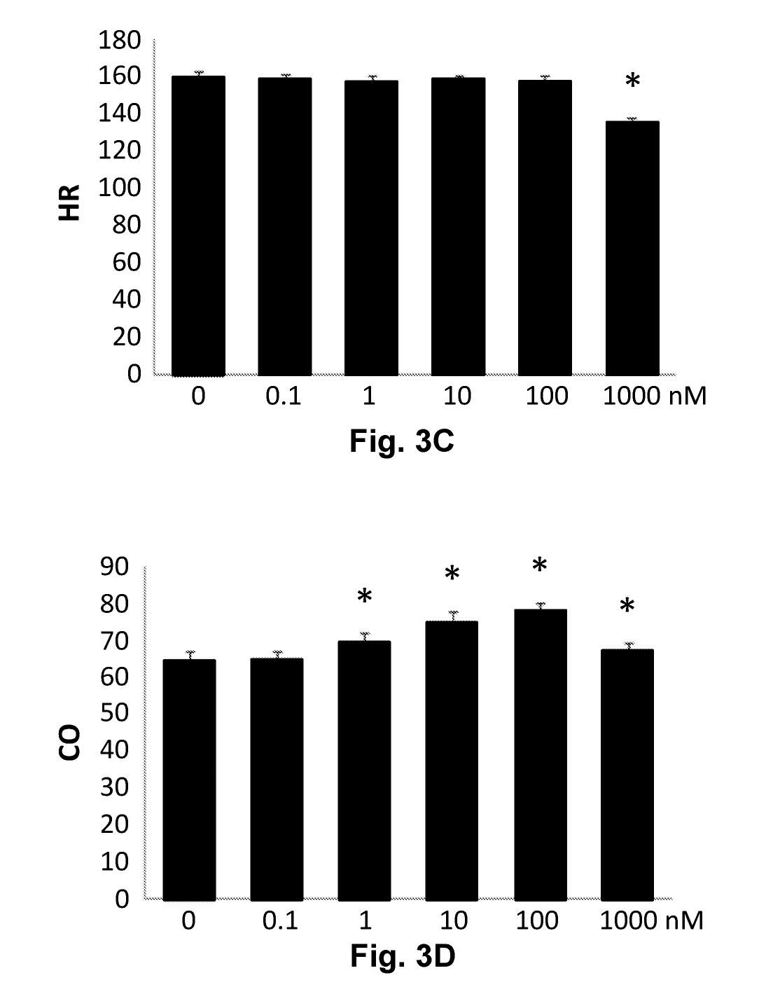

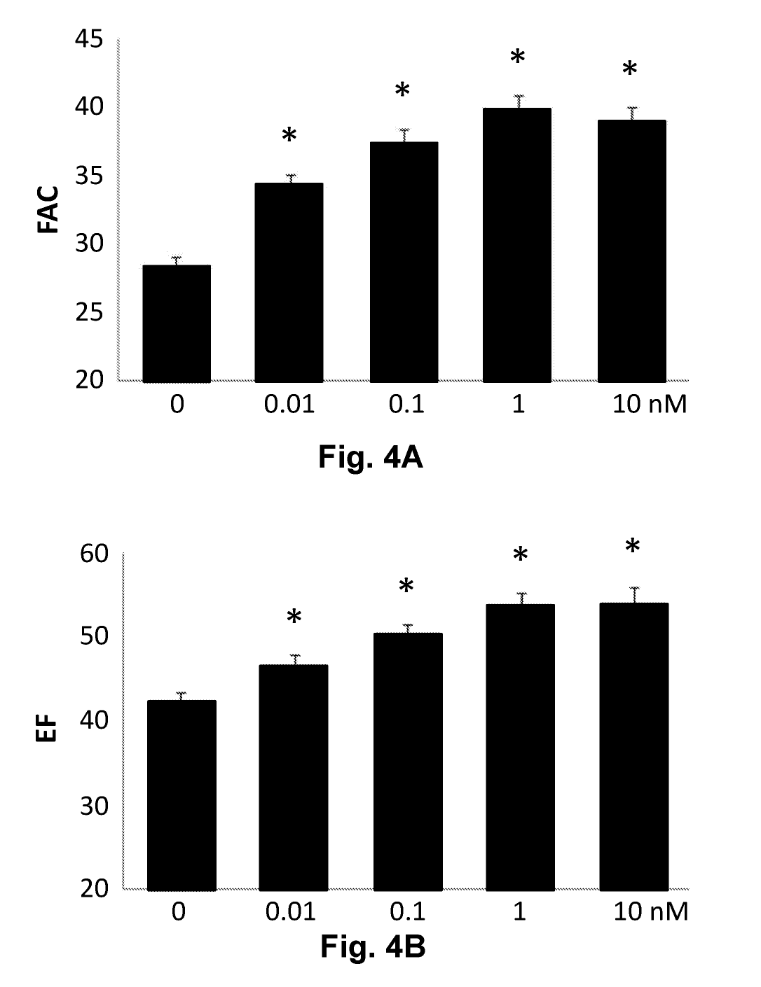

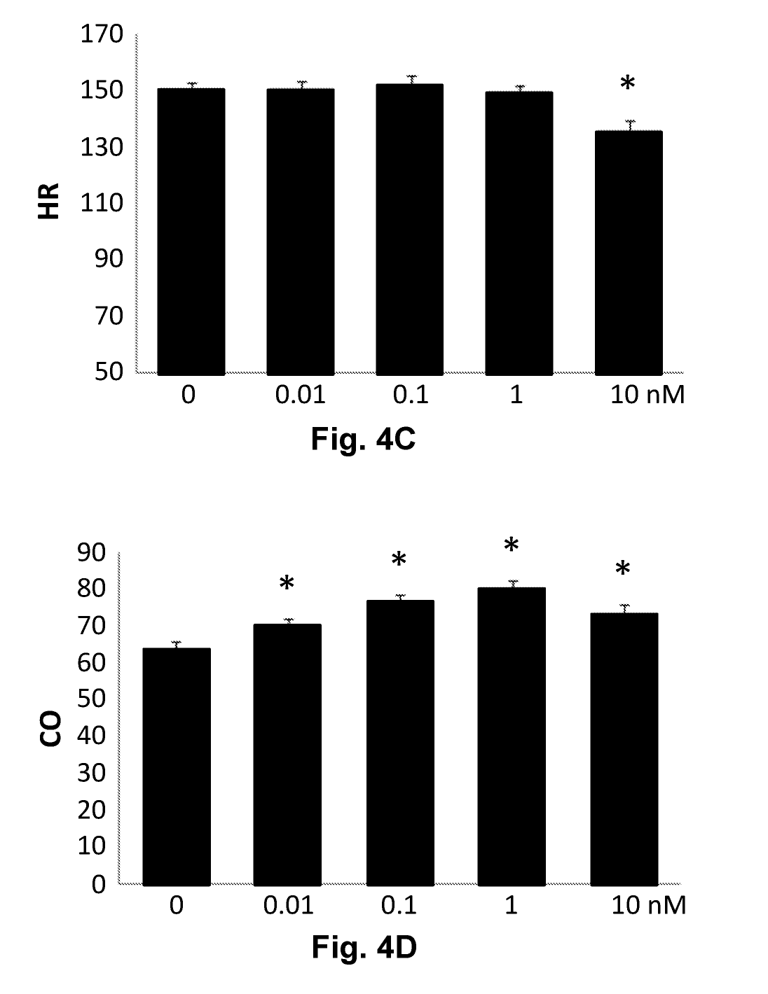

Zebrafish larvae were treated with low concentrations of ouabain (0.05-0.4 nM) for 90 minutes. The exposure to low concentrations of ouabain led to a significant increase in the heart force of contraction in a dose-dependent manner. This was manifested by significant increases in FAC, EF and CO, with a maximal increase of 38±3.68% in CO at 0.2 nM but no change in heart rate (FIGS. 2A-2D). Similar results were obtained in larvae treated with digoxin or bufalin (as shown in FIGS. 3 and 4).

### Example 3

### CS do not Induce an Increase in Heart Contractility in Zebrafish Ace Mutant Larvae

Although some diversity in CS effectiveness was apparent in the experimental system, 1 nM was chosen for the following experiments. At this concentration, the three CS (namely ouabain, digoxin, and bufalin) increased significantly the force of contraction, without affecting heart rate. Hence, as in many other species, the heart of zebrafish larvae respond by increased heart contractility to CS treatment.

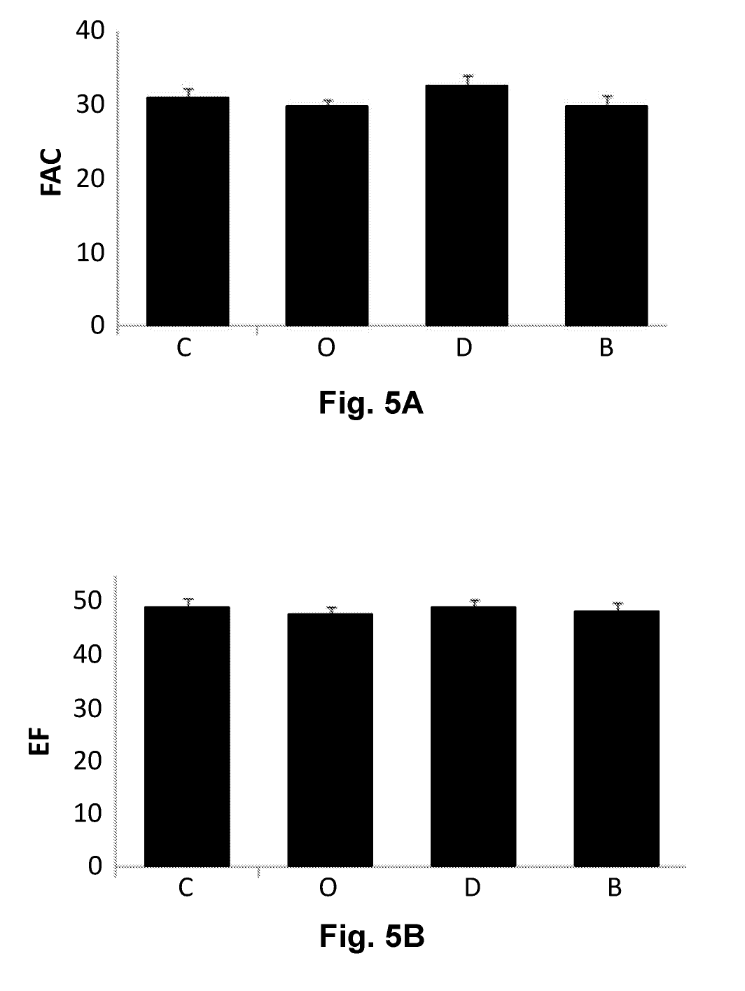

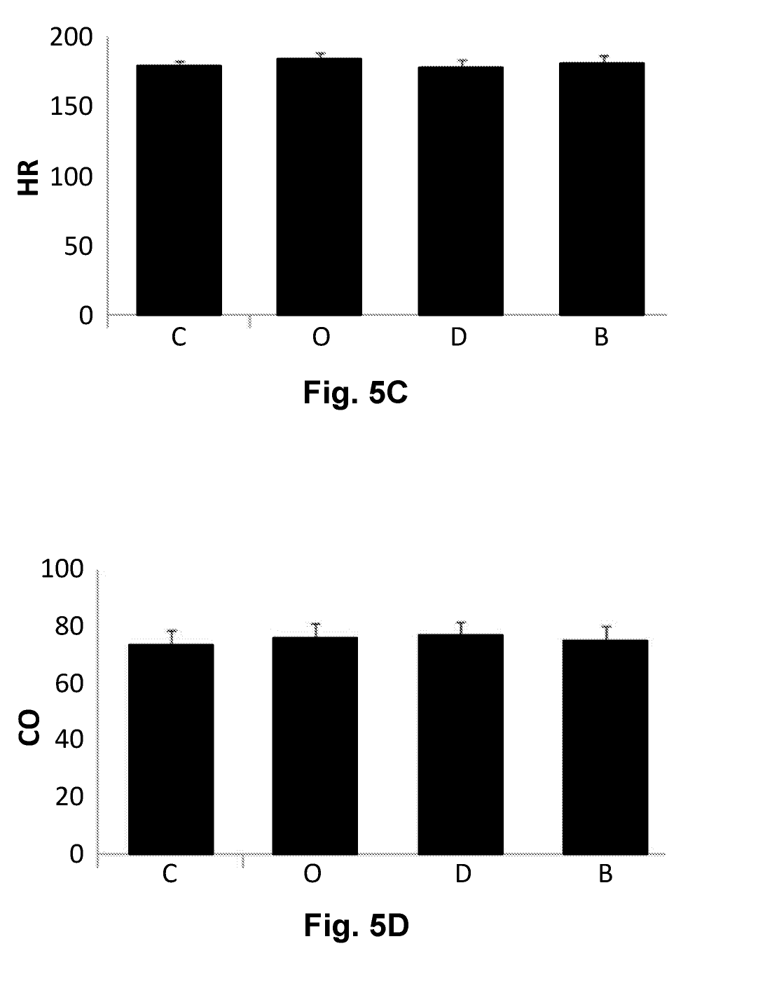

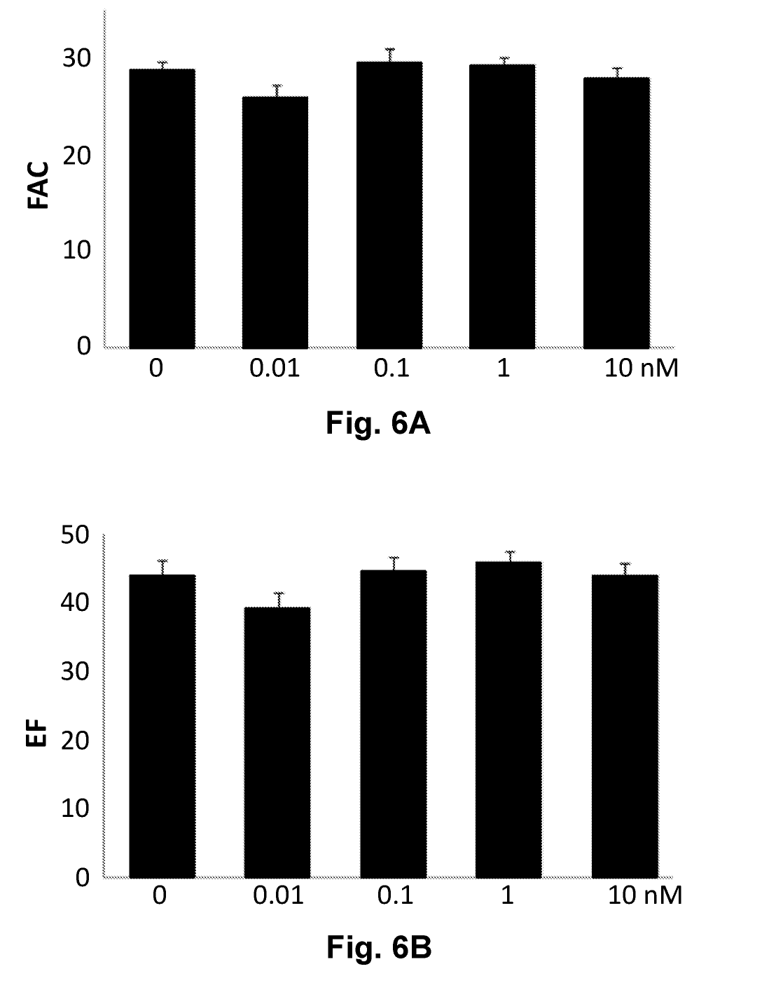

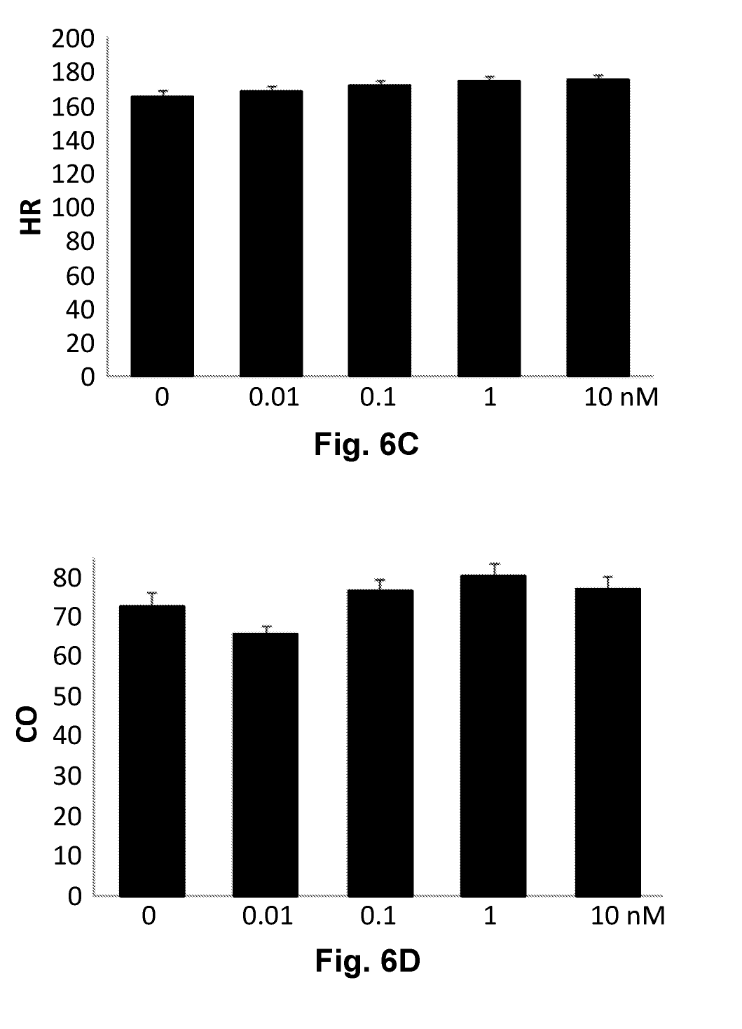

The crucial role of intracellular Ca²⁺ in the CS-induced increase in heart contractility is well established. Hence, in the initial experiments CS action on the zebrafish accordion mutant (acc) was measured. This mutant lacks the activity of the Sarco/Endoplasmic Reticulum Ca(²⁺) ATPase 1a (SERCA1a) isoform exclusively in its muscle cells, inducing slow calcium clearance from the cytoplasm to the sarcoplasmic reticulum. As predicted, exposure of acc larvae to 1 nM ouabain, digoxin or bufalin for 90 minutes had no effect on heart contractility (FIGS. 5A-5D). The same result was obtained at other CS concentrations (0.025-10 nM), which increased contractility in the wt (as shown in FIGS. 6A-6D for ouabain). These results confirm the notion that, as in other species, CS-induced increases in heart contractility in zebrafish larvae largely depend on Ca²⁺ homeostasis.

### Example 4

### CS-Induced Akt and ERK Phosphorylation in Adult Zebrafish Heart In-Vivo

In recent years several laboratories established the effects of CS on the phosphorylation of ERK and Akt proteins in different cells and species. To test this phenomenon in zebrafish, adult specimens were exposed to 1 μM CS for 5 minutes, following which the hearts were removed and the proteins extracted. The phosphorylation states of ERK and Akt in the protein extracts were examined by Western blot analysis. As shown in FIGS. 7A-7D, the addition of ouabain to the swimming media of adult zebrafish resulted in a 130±17.95% and 100±20.06% increase in ERK and Akt phosphorylation in the heart, respectively. Similar results were obtained using digoxin and bufalin (FIGS. 7A-7D). In addition, CS stimulated ERK and Akt phosphorylation also in acc mutants in a manner similar to that seen in the wt (as shown in FIGS. 8A-8D).

### Example 5

### ERK and Src Kinase Inhibitors Attenuate CS-Induce Increases in Heart Contractility in Zebrafish Larvae In-Vivo

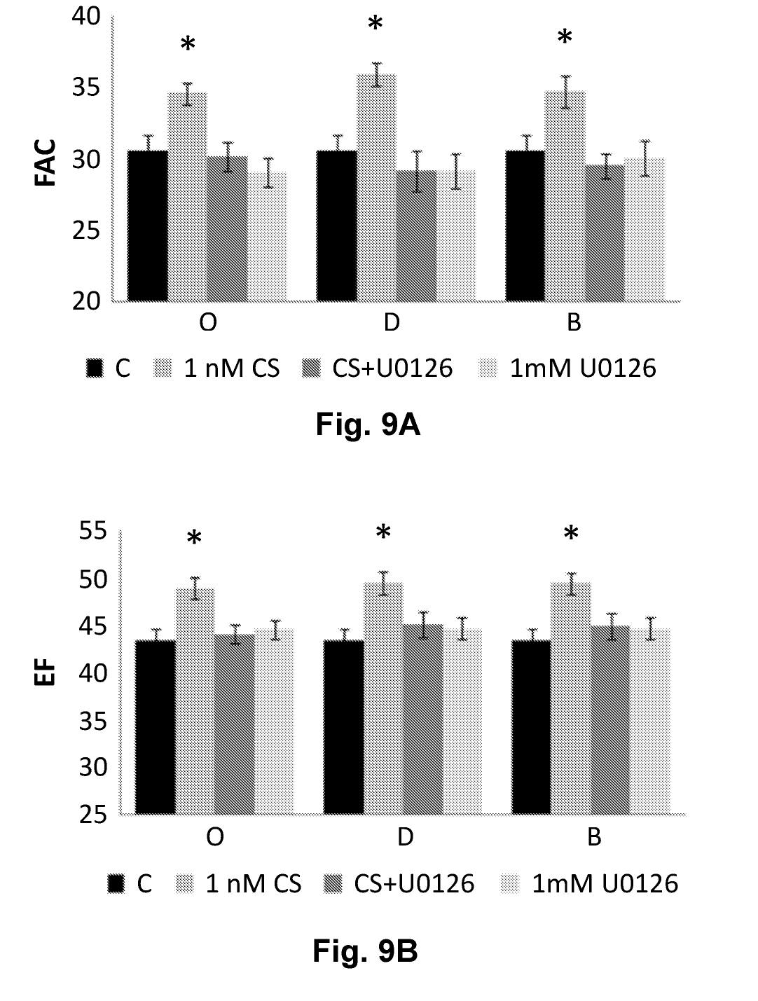

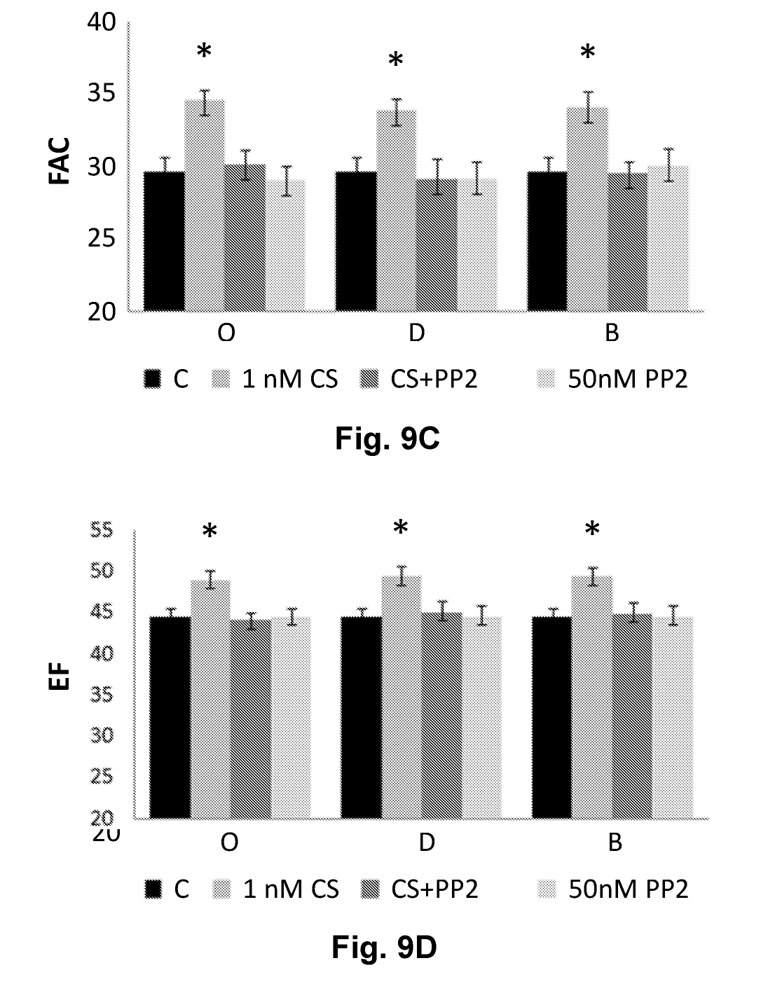

The hypothesis that the Mitogen-activated protein kinases (MAPK) pathway is involved in CS-induced increases in heart contractility was challenged using pharmacological tools. Zebrafish larvae were exposed to ERK or Src kinase inhibitors for 30 minutes, following which CS were added and heart contractility was measured 90 minutes later. As shown in FIGS. 9C and 9D, the inhibitor of Src family kinases, PP2, at a concentration that did not affect heart function (50 nM), completely abolished the CS-induced increase in contractility. Pre-incubation of the larvae with a specific ERK inhibitor, U0126, at a concentration that did not affect basal contractility (1 μM) prevented the CS-induced increase in contractility (FIGS. 9A and 9B). Similar results were obtained with another ERK inhibitor, PD98059 (2′-amino-3′methoxyflavone). Control experiments verified that U0126 and PD98059 inhibit the CS-induced increase in ERK phosphorylation in the adult zebrafish heart.

### Example 6

### Akt Inhibitor Potentiates CS-Induced Increase in Heart Contractility in Zebrafish Larvae In-Vivo

Akt is involved in the PI3K/Akt/mTOR and other signaling pathways and has a key role in multiple cellular processes such as glucose metabolism, apoptosis and cell proliferation in the heart and other organs. To test the possible involvement of Akt activation in CS-induced increase in heart contractility, the effect of Akt inhibition by MK-2206 on zebrafish heart contractility was investigated. The exposure of wt zebrafish larvae for 2 hours to MK-2206 (10 nM) did not affect heart contractility parameters. However, this treatment resulted in a doubling of the CS-induced increase in heart contractility as compared with the CS effect in the absence of the inhibitor (FIGS. 10C and 10D). This augmentation of the response to CS on contractility was apparent in both the FAC and EF determinations and was not accompanied by any effect on heart rate.

The effect of Akt inhibitor was also tested in the acc mutant. Although, as mentioned above, this mutant was not affected by CS at any of the tested concentrations (Example 3), a significant increase in heart contractility caused by 1 nM CS was observed in the presence of Akt inhibitor (FIGS. 10A and 10B).

### Example 7

### ERK and Akt Inhibitors Affect CS-Induced Increase in Contractility in Primary Zebrafish Cardiomyocytes

The results presented in Example 6 regarding the effects of Akt inhibitors on CS-induced increases in heart contractility may have resulted from indirect effects of the inhibitors and/or CS on neuronal or endocrine systems, rather than from direct action on the heart. To test this hypothesis, a similar set of experiments was performed on isolated adult zebrafish cardiomyocytes. As shown in FIGS. 11A-11D and Table 2, treatment of zebrafish cardiomyocytes with 0.1 nM ouabain resulted in a three- to fourfold increase in contractility with a concomitant increase in contractility and relaxation rise time.

Although the addition of 10 nM of ERK inhibitor PD98059 to the cell medium increased contractility and relaxation rise time, it did not affect the amplitude of cell contraction. However, in the presence of the inhibitor, ouabain-induced increases in all contractility parameters were completely prevented (FIGS. 11A and 11B, and Table 2). On the contrary, pre-incubation of cardiomyocytes with 1 nM Akt inhibitor MK-2206, which by itself did not affect any of the contractility parameters, enhanced the ouabain-induced augmentation in contractility manifested by 113, 183 and 169% increases in amplitude, contraction rise time and relaxation rise time, respectively (FIGS. 11C and 11D and Table 2). These results confirm the direct action of ouabain and the ERK and Akt inhibitors on the heart.

In addition, similar to the in-vivo experiments, 0.1 nM ouabain or 1 nM MK-2206, did not affect the amplitude of adult acc mutant primary cardiomyocytes contractility demonstrating the obligatory role of SERCA1a in CS action under normal condition (FIGS. 11E and 11F, and Table 2). However, the addition of ouabain to these cells, in the presence of Akt inhibitor, restored ouabain-induced increase in muscle contraction manifested by 67% increase in the contraction amplitude. These results support the notion that Akt activity plays a negative regulatory role in CS action.

TABLE 2
Contractility parameters of Ouabain-induced increase
in primary cardiomyocyte contractility in the presence
and absence of ERK or Akt inhibitors.

| | | Contraction Rate | Relaxation Rate |
|---|---|---|---|
| | Amplitude | (−dv/dt) | (+dv/dt) |
| Control | 0.14 ± 0.04 | 0.63 ± 0.16 | 0.41 ± 0.09 |
| 0.1 nM Oua | 0.53 ± 0.1# | 3.65 ± 0.38# | 2.92 ± 0.37# |
| 10 nM PD98059 | 0.13 ± 0.01 | 0.87 ± 0.09# | 0.78 ± 0.14# |
| Oua & PD98059 | 0.15 ± 0.02 | 1.06 ± 0.09 | 0.79 ± 0.08 |
| Control | 0.13 ± 0.01 | 0.77 ± 0.14 | 0.50 ± 0.11 |
| 0.1 nM Oua | 0.38 ± 0.05* | 1.84 ± 0.28* | 1.64 ± 0.23* |
| Oua & MK | 0.43 ± 0.04 | 3.37 ± 0.27 | 2.78 ± 0.32 |
| 1 nM MK2066 | 0.19 ± 0.03 | 0.98 ± 0.19 | 0.87 ± 0.20 |

Acc mutant

| | | | |
|---|---|---|---|
| Control | 0.13 ± 0.01 | 1.58 ± 0.29 | 0.56 ± 0.19 |
| 0.1 nM Oua | 0.14 ± 0.03 | 2.43 ± 0.84 | 0.07 ± 0.05* |
| Oua & MK | 0.22 ± 0.02* | 3.65 ± 0.71* | 0.52 ± 0.26 |
| 1 nM MK2066 | 0.15 ± 0.02 | 2.61 ± 0.74 | 0.98 ± 0.34 |

The experiments were performed as described in the legend to FIG. 11. Average twitch amplitude, contraction and relaxation rates were measured in 9 cells in each group.
*Significantly higher than control P < 0.05.

### Example 8

### The Effect of Akt Inhibition on CS-Induced Increase in Contractility in Adult Zebrafish is Ca²⁺ Independent

The novel finding of the potentiation and restoration effects of Akt inhibition on CS-induced increase in heart contractility in wt and acc mutants of zebrafish, respectively, may result from changes in CS-induced Ca²⁺ transients or other Ca²⁺ independent mechanisms. To test the mechanisms involved, basal and CS-induced Ca²⁺ transients were measured in adult wt and acc zebrafish isolated hearts. In the wt zebrafish heart, as expected, the addition of 200 μM ouabain to the medium caused a significant increase in amplitude (27%), and in rise slope (61%) and decay slope (86%) of the Ca²⁺ transients (FIG. 12A and Table 3). Similar changes in the CS-induced Ca²⁺ signals were obtained following pre-incubation of the heart with Akt inhibitor (FIG. 12C and Table 3) indicating that the ouabain effects on Ca²⁺ oscillation are independent of Akt activity. Furthermore, the addition of 200 μM ouabain to acc zebrafish heart did not result in any changes in the Ca²⁺ transients parameters (FIG. 12B and Table 3) showing that SERCA1a activity is required for CS-induced alterations in Ca²⁺ signals. The addition of 200 μM ouabain to acc zebrafish hearts, in the presence of Akt inhibitor (FIG. 12D and Table 3), a condition that induced an increase in heart contractility, also did not cause any changes in Ca²⁺ transient parameters. These Ca²⁺ measurements support the notion that, in zebrafish heart, Akt inhibition does not change ouabain-induced effects on Ca²⁺ transients.

TABLE 3
Effect of ouabain and Akt inhibitor on Ca²⁺ transients in adult zebrafish heart.

| | | Ouabain | MK-2206 | Ouabain & |
|---|---|---|---|---|
| | Control | 200 μM | (1 nM) | MK-2206 |
| Rate | 58 ± 7.4 | 52 ± 6.8 | 48 ± 4.8 | 48 ± 5 |
| (wave/min) | | | | |
| Amplitude | 5.28 ± 0.32 | 6.59 ± 0.71* | 4.8 ± 0.13 | 5.91 ± 0.28*^{#} |
| (Maximal deflection | | | | |
| from baseline) | | | | |
| Area | 2.52 ± 0.28 | 2.94 ± 0.44* | 2.81 ± 0.19 | 2.08 ± 0.23 |
| (Area under the | | | | |
| transient relative | | | | |
| to baseline) | | | | |
| Rise slope | 4.85 ± 1.29 | 7.84 ± 2.02* | 1.65 ± 0.28* | 2.68 ± 0.34*^{#} |
| (+Δ Fluorescence | | | | |
| in the linear | | | | |
| phase/sec) | | | | |
| Decay slope | 10.73 ± 7.54 | 20 ± 3.51* | 4.65 ± 0.54 | 7.87 ± 1.11 |
| (−Δ Fluorescence | | | | |
| in the linear | | | | |
| phase/sec) | | | | |
| Acc mutant | | | | |
| Rate | 31 ± 5 | 35 ± 8.4 | 25 ± 3 | 31 ± 4.4 |
| (wave/min) | | | | |
| Amplitude | 1.79 ± 0.11 | 1.67 ± 0.15* | 1.85 ± 0.05 | 1.76 ± 0.05 |
| (Maximal deflection | | | | |
| from baseline) | | | | |
| Area | 2.64 ± 0.65 | 2.54 ± 0.56 | 2.79 ± 0.23 | 2.92 ± 0.4 |
| (Area under the | | | | |
| transient relative | | | | |
| to baseline) | | | | |
| Rise slope | 0.48 ± 0.07 | 0.47 ± 0.1 | 0.51 ± 0.04 | 0.56 ± 0.05 |
| (+Δ Fluorescence | | | | |
| in the linear | | | | |
| phase/sec) | | | | |
| Decay slope | 1.88 ± 0.33 | 1.92 ± 0.46 | 1.72 ± 0.22 | 1.61 ± 0.19 |
| (−Δ Fluorescence | | | | |
| in the linear | | | | |
| phase/sec) | | | | |

The experiments were performed as described for FIG. 12. Average of Ca²⁺ transient were measured in 10 isolated zebrafish heart in each group.
*Significantly higher than control P < 0.05;
^{#}Significantly higher than MK-2206 P < 0.05

### Example 9

### Improved Efficiency of CS Effects on Heart Contractility by Akt Inhibitor in LAD-Ligated Rats

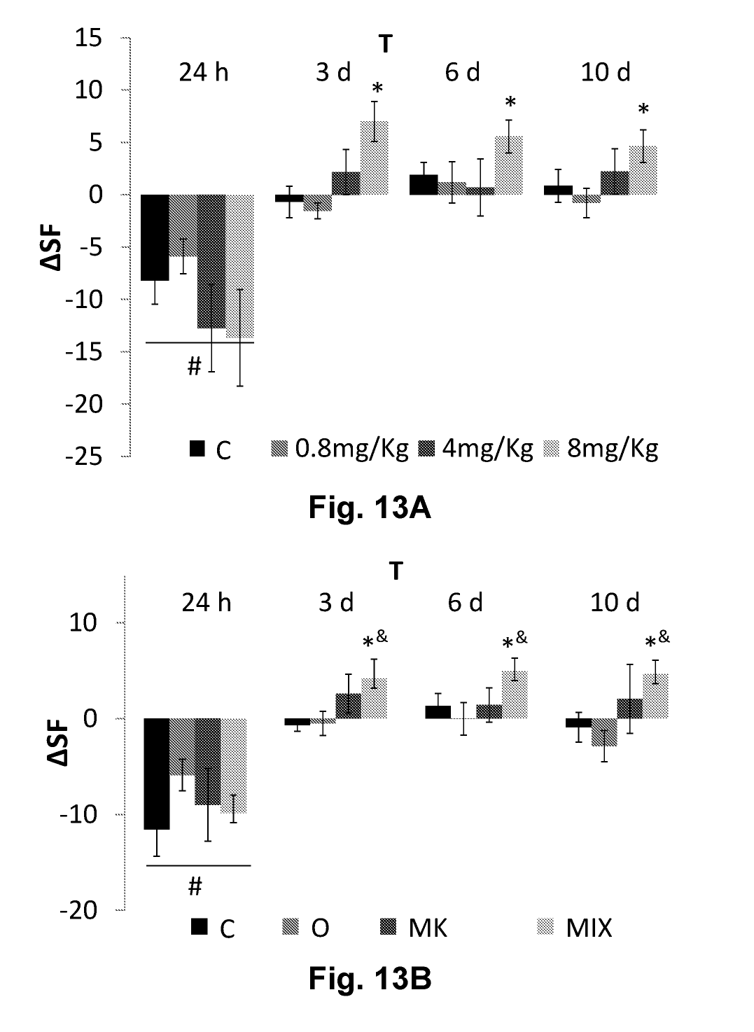

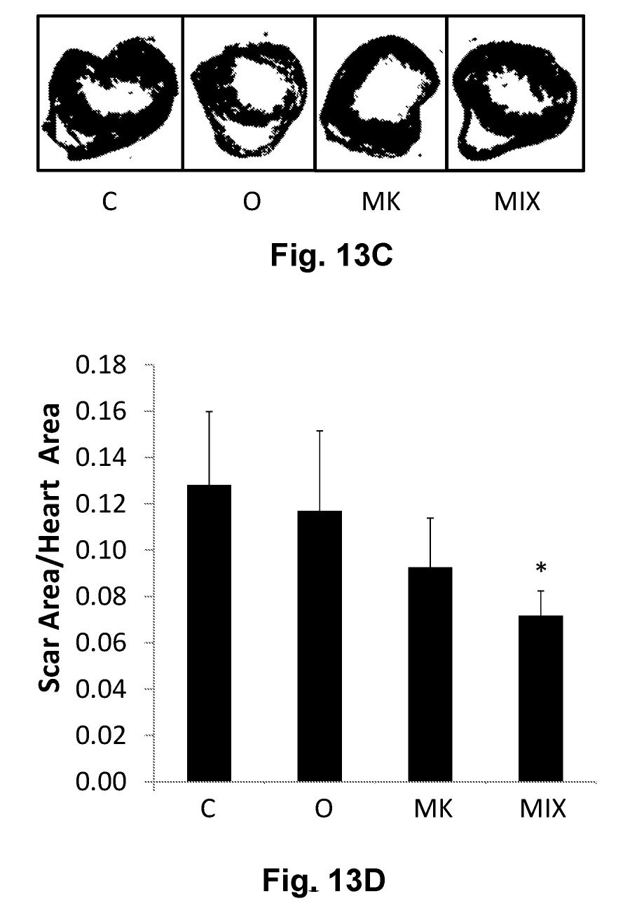

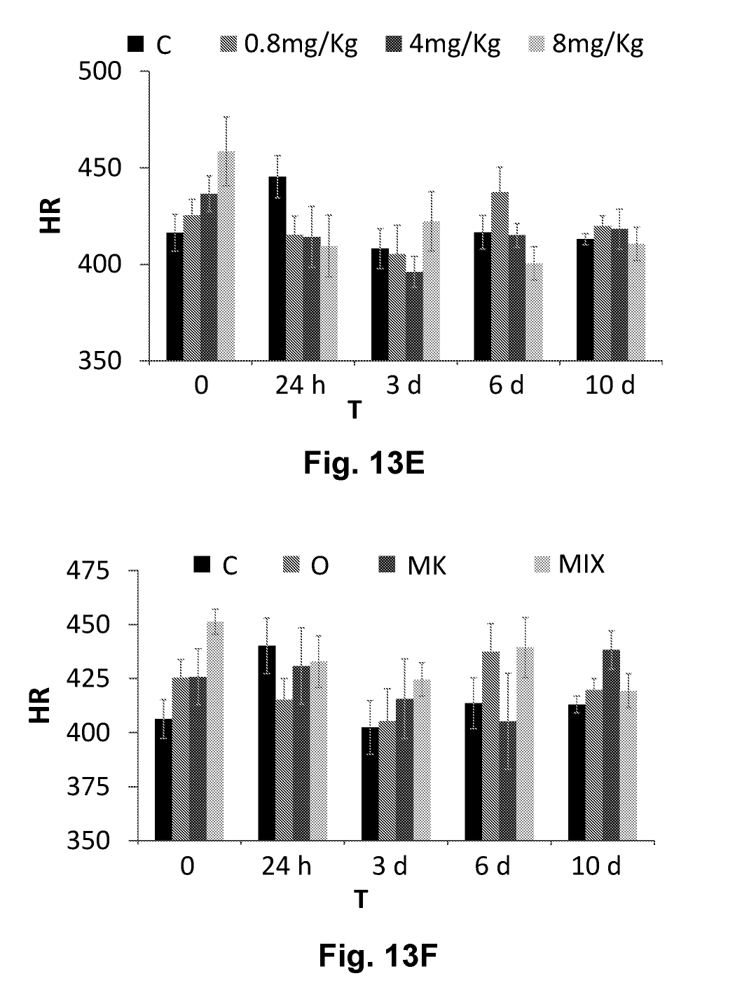

LAD-ligation is an acceptable murine model of myocardial infarction. In this model the left anterior descending artery (LAD) is ligated with one single stitch, forming an ischemia that can be seen immediately. By closing the LAD, no further blood flow is permitted in that area, while the surrounding myocardial tissue is nearly not affected. This surgical procedure imitates the pathophysiological aspects occurring in infarction-related myocardial ischemia and therefore is frequently being used for pharmacological screening for new drugs for the treatment of ischemic heart disease. LAD ligation caused a significant damage to the left ventricle manifested by the significant reduction of shortening fraction values (SF) relative to baseline (FIGS. 13A and 13B), the staining of the area of scare tissue at the end of the experiment (FIG. 13C) and the increase in troponin T (Trop-T) and creatine phosphokinase (CPK) in the circulation, determined at 24 hours post MI (Table 4). Changes in plasma electrolytes, namely Na⁺ and K, and uric acid are similar to those reported previously. These experiments verified the damage induced by the ligation.

Preliminary experiments were designed to determine ouabain dose to be used in the following experiments. To this end, the effects of ouabain at 0.8, 4 and 8 mg/kg/day on heart contractility following LAD-ligation were measured. As shown in FIG. 13A, only the high dose of 8 mg/kg/day significantly increased the SF of the heart starting at the 3^{rd} day of treatment. Therefore, the beneficial effect of ouabain-MK-2206 combination was tested at the dose of 0.8 mg/kg/day, which by itself did not increase SF following LAD-ligation.

The effects of the treatment of rats with ouabain (0.8 mg/kg/day), MK-2206 (12 mg/kg/day) and combination of the two drugs on heart contractility are shown in FIG. 13B. Administration of ouabain at this dose, MK-2206 or saline to the rats did not result in SF improvement relative to 24 hours post MI at any tested time point. On the contrary, treatment of rats with the combination of ouabain and MK-2206, significantly improved SF at 3, 6 and 10 days post MI as compared to SF values at 24 hours post MI. Importantly, the improvement in SF following the combined treatment was also significant in comparison to that seen in rats treated with ouabain alone.

The beneficial effect of the combined treatment of ouabain together with MK-2206 is also manifested in the scar area observed 10 days following LAD-Ligation (FIGS. 13C and 13D). The combined treatment, unlike the treatment by ouabain or MK-2206 alone, significantly reduced the heart scar area resulting from the LAD ligation. Heart rate measurements of rats during the experiments revealed that LAD-ligation, ouabain, MK-2206 and combined treatment by the two drugs did not affect significantly heart rate 24 hours, 3 and 6 days post MI (FIGS. 13E and 13F).

TABLE 4
Biochemical changes following lad-ligation and the different treatments.

| | CPK_{(unit/L)} | Trop-T_{(ng/ml)} | Na⁺_{(mM)} | K⁻_{(mM)} | Uric Acid_{(μM)} |
|---|---|---|---|---|---|
| Baseline | 265.27 ± 41.57 | 0.006 ± 0.0003 | 140.72 ± 0.67 | 5.24 ± 0.11 | 82.43 ± 6 |
| 24 hrs. post MI | 2183.25 ± 472.7* | 1.62 ± 0.33* | 134.58 ± 0.75* | 9.6 ± 0.49* | 271.69 ± 22.57* |
| 10 day post MI | | | | | |
| Ctrl | 430 ± 98.1 | 1.33 ± 0.1 | 139.4 ± 1.74 | 6.12 ± 0.56 | 50.5 ± 9.77 |
| Oua | 616.6 ± 197.6 | 0.24 ± 0.17^{#} | 134.66 ± 1.47 | 6.56 ± 0.57 | 72.2 ± 21.73 |
| MK-2206 | 363.5 ± 88.59 | 0.07 ± 0.06^{#} | 140.5 ± 1.25 | 5.8 ± 0.58 | 62.5 ± 24.13 |
| MIX | 387.8 ± 73.34 | 0.41 ± 0.21^{#} | 138.6 ± 1.3 | 6.28 ± 0.3 | 68.5 ± 12.18 |

Blood samples were collected from rat eye at baseline and at 24 hours post LAD-ligation. The plasmas of 11 animals were tested for the indicated biomarkers of MI.
*Significantly different from baseline, (P < 0.005),
^{#}Significantly different from control group, (P < 0.005).

1. A method of treating a cardiovascular disease or disorder comprising administering a pharmaceutical combination comprising a cardiac steroid (CS) and at least one PI3K/Akt/mTOR inhibitor to a subject in need thereof.

2. The method according to claim 1 wherein the CS is selected from:
3-(alpha-L-Rhamnopyranosyloxy)-1beta,5beta,11alpha,14,19-pentahydroxy-5beta-card-20(22)-enolide (ouabain);
4-[(3 S,5R,8R,9S,10S,12R,13S,14S)-3-[(2S,4S,5R,6R)-5-[(2S,4S,5R,6R)-5-[(2S,4 S, 5R, 6R)-4,5-dihydroxy-6-methyl-oxan-2-yl]oxy-4-hydroxy-6-methyl-oxan-2-yl]oxy-4-hydroxy-6-methyl-oxan-2-yl]oxy-12,14-dihydroxy-10,13-dimethyl-1,2,3,4,5,6,7,8,9,11,12,15,16,17-tetradecahydrocyclopenta[a]phenanthren-17-yl]-5H-furan-2-one (digoxin);
5-[(3 S,5R,8R,9S,10S,13R,14S,17R)-3,14-dihydroxy-10,13-dimethyl-1,2,3,4,5,6,7,8,9,11,12,15,16,17-tetradecahydrocyclopenta[a]phenanthren-17-yl]pyran-2-one (bufalin);
(3β,5β)-3-{[3-O-Acetyl-2,6-dideoxy-β-D-ribo-hexopyranosyl-(1->4)-2,6-dideoxy-β-D-ribo-hexopyranosyl-(1->4)-2,6-dideoxy-β-D-ribo-hexopyranosyl]oxy}-14-hydroxycard-20(22)-enolide (acetyldigitoxin);
(3β,5β,12β)-3-{[3-O-Acetyl-2,6-dideoxy-β-D-ribo-hexopyranosyl-(1->4)-2,6-dideoxy-β-D-ribo-hexopyranosyl-(1->4)-2,6-dideoxy-β-D-ribo-hexopyranosyl]oxy}-12,14-dihydroxycard-20(22)-enolide (acetyldigoxin);
5,14-dihydroxy-3-(5-hydroxy-4-methoxy-6-methyloxan-2-yl)oxy-13-methyl-17-(5-oxo-2H-furan-3-yl)-2,3,4,6,7,8,9,11,12,15,16,17-dodecahydro-1H-cyclopenta[a]phenanthrene-10-carbaldehyde (cymarin);
(3β,5β)-3-{[2,6-Dideoxy-β-D-ribo-hexopyranosyl-(1->4)-2,6-dideoxy-β-D-ribo-hexopyranosyl-(1->4)-2,6-dideoxy-β-D-ribo-hexopyranosyl]oxy}-14-hydroxycard-20(22)-enolide (digitoxin);
(3β,5β)-3,14-Dihydroxycard-20(22)-enolide (digitoxigenin);
(3β,5β,12β)-3,12,14-Trihydroxycard-20(22)-enolide (digoxigenin);
(3β,5β,12β)-3-{[2,6-Dideoxy-4-O-methyl-13-D-ribo-hexopyranosyl-(1->4)-2,6-dideoxy-β-D-ribo-hexopyranosyl-(1->4)-2,6-dideoxy-β-D-ribo-hexopyranosyl]oxy}-12,14-dihydroxycard-20(22)-enolide (metildigoxin);
(3 S,5 S,8R,9S,10S,13R,14S,17R)-5,14-Dihydroxy-13-methyl-17-(5-oxo-2,5-dihydro-3-furanyl)-3-{[(2R,3R,4R,5R,6R)-4,5,6-trihydroxy-3-{[(2S,3R,4S,5S,6R)-3,4,5-trihydroxy-6-(hydroxymethyl)tetrahydro-2H-pyran-2-yl]oxy}tetrahydro-2H-pyran-2-yl]oxy}hexadecahydro-1 OH-cyclopenta[a]phenanthrene-10-carbaldehyde (neoconvalloside);
(3β,5β)-3-{[2,6-Dideoxy-4-O-(β-D-glucopyranosyl)-3-O-methyl-3-D-ribo-hexopyranosyl]oxy}-5,14-dihydroxy-19-oxocard-20(22)-enolide (k-strophanthin);
(3β,5β)-3,5,14-Trihydroxy-19-oxocard-20(22)-enolide (k-strophanthidin);
5-[(5R,8R,9S,10S,13S,14S,17S)-10,13-dimethyl-2,3,4,5,6,7,8,9,11,12,14,15,16,17-tetradecahydro-1H-cyclopenta[a]phenanthren-17-yl]pyran-2-one (bufadienolide);
5-[(3 S,8R,9S,1 OR, 13R, 14S, 17R)-14-Hydroxy-10,13-dimethyl-3-((2R,3R,4R,5R,6R)-3,4,5-trihydroxy-6-methyltetrahydro-2H-pyran-2-yloxy)-2,3,6,7,8,9,10,11,12,13,14,15,16,17-tetradecahydro-1H-cyclopenta[a]phenanthren-17-yl]-2H-pyran-2-one (proscillaridin);
(1β,3β,5β,11α)-1,3,5-(ethylidynetris(oxy)-11,14-dihydroxy-12,19-dioxobufa-20,22-dienolide (daigremontianin); and
(3β,5β,15β)-3,5-Dihydroxy-14,15-epoxybufa-20,22-dienolide (marinobufagenin).

3. The method according to claim 1 wherein the CS is ouabain, digoxin, or bufalin.

4. The method according to claim 1 wherein the PI3K/Akt/mTOR inhibitor is an Akt inhibitor selected from:
8-[4-(1-Aminocyclobutyl)phenyl]-9-phenyl[1,2,4]triazolo[3,4-f][1,6]naphthyridin-3(2H)-one dihydrochloride (MK-2206 2HCl);
1,1-dimethyl-4 [(octadecyloxy)hydroxyphosphinyl]oxy]-piperidinium inner salt, KRX-0401 (perifosine);
4-[2-(4-amino-1,2,5-oxadiazol-3-yl)-1-ethyl-7-[[(3 S)-piperidin-3-yl]methoxy]imidazo[4,5-c]pyridin-4-yl]-2-methylbut-3-yn-2-ol (GSK690693);
(2 S)-2-(4-Chlorophenyl)-1-{4-[(5R,7R)-7-hydroxy-5-methyl-6,7-dihydro-5H-cyclopenta[d]pyrimidin-4-yl]-1-piperazinyl}-3-(isopropylamino)-1-propanone (GDC-0068, ipatasertib);
4-amino-N-[(1 S)-1-(4-chlorophenyl)-3-hydroxypropyl]-1-(7H-pyrrolo[2,3-d]pyrimidin-4-yl)piperidine-4-carboxamide (AZD5363);
2-amino-8-[4-(2-hydroxyethoxy)cyclohexyl]-6-(6-methoxypyridin-3-yl)-4-methylpyrido[2,3-d]pyrimidin-7-one (PF-04691502);
4-(4-chlorophenyl)-4-[4-(1H-pyrazol-4-yl)phenyl]piperidine (AT7867);
5-Methyl-1-(β-D-ribofuranosyl)-1,5-dihydro-1,4,5,6,8-pentaazaacenaphthylen-3-amine (Triciribine);
4-(4-Chlorobenzyl)-1-(7H-pyrrolo[2,3-d]pyrimidin-4-yl)-4-piperidinamine (CCT128930);
(2S)-1-[5-(3-methyl-2H-indazol-5-yl)pyridin-3-yl]oxy-3-phenylpropan-2-amine (A-674563);
4-dodecyl-N-(1,3,4-thiadiazol-2-yl)benzenesulfonamide (PHT-427);
3-[1-[[4-(7-phenyl-3H-imidazo[4,5-g]quinoxalin-6-yl)phenyl]methyl]piperidin-4-yl]-1H-benzimidazol-2-one (Akti-1/2);
N-[(2S)-1-amino-3-(3-fluorophenyl)propan-2-yl]-5-chloro-4-(4-chloro-2-methylpyrazol-3-yl)thiophene-2-carboxamide (GSK2110183, afuresertib);
(1 S)-2-amino-1-(4-chlorophenyl)-1-[4-(1H-pyrazol-4-yl)phenyl]ethanol);
Miltefosine (hexadecyl 2-(trimethylazaniumyl)ethyl phosphate (AT13148);
2-(4-hydroxy-3-prop-2-enylphenyl)-4-prop-2-enylphenol (Honokiol);
2,6,7,8,9,10-hexahydro-10-[(2-methylphenyl)methyl]-7-(phenylmethyl)-imidazo[1,2-a]pyrido[4,3-d]pyrimidin-5(3H)-one (TIC10 Analogue);
2,4,6,7,8,9-hexahydro-4-[(2-methylphenyl)methyl]-7-(phenylmethyl)-imidazo[1,2-a]pyrido[3,4-e]pyrimidin-5(1H)-one (TIC10); and
ethyl-3-aminobenzoate methanesulfonate salt (MS-222).

5. The method according to claim 4 wherein the Akt inhibitor is perifosine, miltefosine, or MS-222.

6. The method according to claim 1 wherein the combination further comprises at least one additional therapeutic agent.

7. (canceled)

8. The method according to claim 1, wherein the cardiovascular disease or disorder is a coronary heart disease, myocardial infarction, heart failure, chronic atrial fibrillation, acute atrial fibrillation, peripheral arterial disease, rheumatic heart disease, or congenital heart disease.

9. The method according to claim 1, wherein said combination is administered simultaneously, sequentially or separately.

10-16. (canceled)

17. A method for improving efficacy of the treatment of a cardiovascular disease or disorder with at least one PI3K/Akt/mTOR inhibitor comprising administering a combination comprising a CS and at least one PI3K/Akt/mTOR inhibitor to a subject in need thereof.

18. A kit comprising: (a) a first container with a CS; (b) a second container with PI3K/Akt/mTOR inhibitor; optionally (c) a third container with a third pharmaceutical formulation; and (d) label or package insert with instructions for treating a cardiovascular disease or disorder.

19. The kit according to claim 18, wherein the CS and the PI3K/Akt/mTOR inhibitor are provided as different dosage forms, each one in a suitable carrier.

20. The method according to claim 1, wherein the CS is digoxin, which is administered at a dose of 5 mcg per day or less.

21. A dosage form of digoxin comprising 0.1-5 mcg for treating a cardiovascular disease or disorder in combination with a PI3K/Akt/mTOR inhibitor.
